(19) United States
(12) Patent Application Publication
(10) Pub. No.: US 2019/0331236 A1
(43) Pub. Date: Oct. 31, 2019

(54) ROTARY VALVE

(71) Applicant: University of Iowa Research Foundation, Iowa City, IA (US)

(72) Inventors: Gurjap Singh, Iowa City, IA (US); Albert Ratner, Iowa City, IA (US)

(21) Appl. No.: 16/312,689

(22) PCT Filed: Jun. 28, 2017
(86) PCT No.: PCT/US2017/039683

§ 371 Date: Dec. 21, 2018

Related U.S. Application Data
(60) Provisional application No. 62/355,718, filed on Jun. 28, 2016.

(51) Int. Cl. F16K 3/08; A61M 1/10; A61F 2/24; A61M 1/36

(52) U.S. Cl. CPC F16K 3/085; A61M 1/1087; A61M 1/1005; A61F 2/2403; A61M 1/3666; A61M 1/1036

(57) ABSTRACT

A rotary valve includes at least one rotatable valve member configured to be operatively connected to and rotate relative to a fluid conduit. The rotatable valve member includes at least one aperture. The rotatable valve member is capable of being positioned in a plurality of positions relative to the conduit. The position of the at least one first aperture of the rotatable valve member controls fluid flow through the rotary valve, and thereby through the conduit.

### CLAIM OF PRIORITY

This application claims the benefit of U.S. Provisional Patent Application Ser. No. 62/355,718, filed on Jun. 28, 2016, the benefit of priority of which is claimed hereby, and which is incorporated by reference herein in its entirety.

### DESCRIPTION OF THE DRAWINGS

In the drawings, which are not necessarily drawn to scale, like numerals may describe similar components in different views. Like numerals having different letter suffixes may represent different instances of similar components, sub-components of a larger logical or physical system, or the like. The drawings illustrate generally, by way of example, but not by way of limitation, various examples described in the present disclosure.

FIG. 1 depicts an example rotary valve in accordance with this disclosure.

FIGS. 2A-2C depict another example valve in accordance with this disclosure.

FIG. 3 depicts two rotary valves in accordance with FIGS. 2A-2C employed in a flow splitting or flow mixing application (depending upon the direction of fluid flow).

FIGS. 4A and 48 depict another example rotary valve.

FIG. 5 depicts another example rotary valve.

FIG. 6 schematically depicts chlorine level control system for a swimming pool including at least one rotary valve in accordance with this disclosure.

FIG. 7 depicts another example rotary valve in accordance with this disclosure.

FIG. 8 depicts another example rotary valve.

FIGS. 9A and 9B depict another example rotary valve.

FIGS. 10A-12D depict another example rotary valve in accordance with this disclosure.

FIGS. 13A and 13B depict another example rotary valve.

FIGS. 14A and 14B schematically depict an internal combustion engine including two rotary valves in accordance with this disclosure.

FIGS. 15A and 1513 depict the outer rotor of the valve of FIGS. 14A and 14B in more detail.

FIGS. 16A and 16B depict the inner rotor of the valve of FIGS. 14A and 14B in more detail.

FIGS. 17A-17D depict the rotary valve of FIGS. 14A and 14B including a valve body and the inner rotor and outer rotors of FIGS. 15A-16B.

FIG. 18A depicts another example rotary valve in accordance with this disclosure.

FIG. 18B depicts the inner and outer rotor of the rotary valve of FIG. 18A.

### DETAILED DESCRIPTION

There are a variety of practical applications for which pulsatile or pulsed fluid flow is required or beneficial. Pulsatile flow (PF), as used in this disclosure, is a stream or flow of fluid that flows at other than a substantially constant velocity, volume/mass rate, and/or pressure. There are a number mechanisms by which PF can be produced. For example, a pulsatile fluid pump can be designed to draw fluid from a source and deliver the fluid in a pulsed flow. However, pulsatile pumps tend to be more complex, larger, more challenging to control (if actively controllable at all) and likely more expensive to produce than a constant flow fluid pump.

Examples according to this disclosure are directed to rotary valves that are capable of delivering pulsatile flow of a fluid at a variety of simply and reliably controlled frequencies, which frequencies can be selected depending upon the intended application. In one example, a rotary valve includes at least one rotatable valve member configured to be operatively connected to and rotate relative to a fluid conduit. The rotatable valve member includes at least one aperture. The rotatable valve member is capable of being positioned in a plurality of positions relative to the conduit. The position of the at least one first aperture of the rotatable valve member controls fluid flow through the rotary valve, and thereby through the conduit. In some cases, a single operational parameter of the valve, angular velocity, can be controlled to deliver a target frequency PF. The valve can rotate continuously or periodically. The valve can rotate in a single or two directions. The valve can also rotate at a constant or variable velocity.

Rotary valves in accordance with this disclosure may be employed to block and let pass fluid flow in a pulsed or constant flow. Additionally, rotary valves in accordance with this disclosure may be employed to control mass/volume flow rate a fluid by varying the cross section of a fluid conduit through which the fluid flows.

In one example, a rotary valve includes a rotating disc. The disc includes at least one aperture and is configured to be operatively coupled to a fluid conduit. The disc may be, for example, arranged with a major face approximately perpendicular to a longitudinal axis of the fluid conduit. The disc is arranged so that the center and axis of rotation of the disc is offset from the longitudinal axis of the fluid conduit. The disc is configured to rotate continuously or periodically to arrange the at least one aperture in a variety of positions. As the at least one aperture rotates into alignment with the fluid conduit, fluid can flow there through. As the at least one aperture rotates out of alignment with the fluid conduit, fluid flow there through is stopped.

As an example application that may benefit from PF, various biological processes in the human body rely on consistent, pulsatile blood flow to be effective. The pressure and velocity of blood flow in the body is controlled by the heart. People who suffer from heart failure require implantable devices to assist in maintaining healthy blood flow. These can be, as examples, a Total Artificial Heart (TAH) or a Ventricular Assist Device (VAD). Research shows CF devices cause problems such as hemolysis, gastrointestinal bleeding, and aortic insufficiency, and cannot be considered as the best possible solutions for patients already suffering from heart failure. Example rotary valves in accordance with this disclosure can capable of producing strong, mathematically accurate PF at required frequencies to mimic the human heart, while being compact and reliable.

5 million Americans currently live with heart failure, and 670,000 new cases are diagnosed each year. Only 2000 advanced heart failure patients receive transplants each year. More than 250,000 advanced heart failure patients have no viable treatment option, and up to 100,000 of these could benefit from a Left Ventricular Assist Device or LVAD. At least some example rotary valves in accordance with this disclosure may make TAH and LVAD systems a long-term solution and a potential alternative for heart transplant surgery.

Example rotary valves in accordance with this disclosure can produce pressure and/or velocity pulses according to a wide variety of mathematical functions (for example, a sine wave). In one example, a rotary valve includes parallel discs, at least one of which is rotatable. The discs are configured to be operative coupled to and arranged at least partially within a fluid conduit. Each disc includes slots. By controlling the shape of the slot and the angular velocity of one or both of the discs it is possible to attain fluid flows that fit virtually any desired velocity or pressure field. In some cases, both discs rotate. In another example, one disc is a rotor and the other a stator.

The valve can be hermetically sealed and can be fitted downstream of a standard axial/centrifugal or other constant flow fluid pump. Simple control logic and easy operation (control of disc speeds) may produce a better response time compared to existing devices delivering PF. The simplicity of the design and operation parameters can increase durability and reliability. Additionally, the valve rotor (and/or stator) design can be optimized to reduce drag and flow separation and generally to reduce and control for fluid pressure loss.

Rotary valves in accordance with this disclosure can be actively or passively actuated. For example, a rotary valve can include one rotor and one stator. At least partially surrounding the rotor can be an electric motor armature and connected to or embedded in the rotor can be a magnetic material. By running a constant or variable electrical current through the armature, the rotor can be rotated continuously or periodically, in one or two directions, to position the rotor in various positions relative to the stator at various constant and/or variable velocities.

In another example, a rotary valve includes two rotors, associated and operatively arranged with each of which is an electric motor armature. Each or both of the armatures can be energized to actuate the rotors to rotate into various positions relative to one another. Simple control of current delivered to the armatures will cause the rotors to rotate at constant or variable angular velocities and thereby deliver simple or complex PF profiles.

Although some examples described herein employ an electrical or electromagnetic actuation mechanism, other types of actuators can be employed to actuate rotary valves in accordance with this disclosure, including, for example, mechanical, electro-mechanical, hydraulic and/or pneumatic actuators. In one example, a rotor of a rotary valve is actuated by a mechanical gear mechanism. For example, the rim or outer periphery of the rotor can include a plurality of gear teeth circumferentially disposed thereabout. A drive gear can be arranged to engage the rotor to actuate the rotor and cause it to rotate at a constant or variable angular velocity.

Additionally, more complex gear mechanisms can be employed to provide increased control and variability to the operation of the valve. For example, the rotor can be operatively connected to a gear box including one or more gears and/or gear trains to provide multiple gear ratios in order to vary the angular velocity of the rotor of the valve.

In another example, a rotary valve is passively actuated. For example, a rotary valve can be arranged at least partially within and operatively connected to a fluid conduit. The rotary valve can include a rotor and a stator. The rotor can be configured to be actuated to rotate relative to the stator by a pressure differential on either side of the valve. Passively actuated valves in accordance to this disclosure can be configured to respond (e.g., actuate) to fluid flow fields applied to them. For example, a rotary valve in accordance with this disclosure can be employed as an artificial heart valve and the blood flow through the passive artificial valve causes the valve to open or close.

Another example includes a turbine upstream of an example rotary valve, which turbine is as the flow passes through it. The turbine can be coupled via a shaft to a rotor of the valve. The fluid flow, in this case, causes the turbine to rotate and, in turn, the turbine rotates the valve.

Another example application that may benefit from rotary valves in accordance with this disclosure is internal combustion (IC) engines. Modern IC engine valves employ a tapered valve stem translating axially in a valve body, where the valve stem can move relative to the valve body using cam or solenoid actuation in order to create a gap between the valve stem and the valve body. Such an arrangement can be used then to let in air or air-fuel mixture, or to let out exhaust gases, at appropriate times in the cycle of the engine. Although the idea is to generally facilitate flow or to block it, this technique, by design, puts a large shaft in the path of fluid movement when the overall intent is for the fluid to flow very quickly in or out of the combustion chamber or some other fluidic flow control environment.

Moreover, there is a need by automobile manufacturers, to extract the most energy out of the fuel per cycle, either to improve performance or efficiency, or to conform to ever more stringent environmental regulations. Variable valve timing (VVT) has helped in this regard by varying the timing of valve opening/closing between different engine operating conditions, e.g. revolutions per minute (RPMs). However, VVT typically requires every valve to be fitted with a solenoid, and all solenoids to be controlled with electronic logic. Despite these advances, the design of conventional valves may limit efficiency and performance.

In one example rotary valve in accordance with this disclosure, the valve includes two valve members, at least one of which is rotatable and both of which are operative coupled to the header of an IC engine at the top of one cylinder. The valve can be a fuel input or exhaust valve.

A first one of the two valve members includes a stem, one end of which includes an at least partially circular disc extending radially outward from the stem. The disc of the first valve member includes at least one aperture or a cutout in the disc. The second valve member includes an annular stem, one end of which includes an at least partially circular disc extending radially outward from the annular stem. The disc of the second valve member includes at least one aperture or a cutout in the disc. The stem of the first valve member is received within the annular stem of the second valve member such that the disc of the first valve member is adjacent the disc of the second valve member.

The valve is arranged to position the discs of the first and second valve members in an aperture in the IC engine cylinder. For example, the discs may be positioned in a hole in the cylinder with the major faces of the discs approximately perpendicular to the axis of the hole. At least one of the first and second valve members is rotatable relative to the other to position the apertures/cutouts of the discs of the first and second valve members in a variety of positions. As the apertures/cutouts in the discs rotate into alignment with the aperture in the cylinder, fuel, air-fuel mixture, or exhaust can enter or exhaust from the cylinder. As the at least one aperture rotates out of alignment with the aperture in the cylinder, flow there through is stopped. The shape and positioning of the disc apertures/cutouts and valve member(s) angular velocity can be varied for engine-specific spray tailoring to improve performance and economy through better mixing, stratified flow and stratified combustion.

In some examples, rotary valves in accordance with this disclosure can be configured to split a stream of fluid flow into two or more branches, or fluid circuits. Additionally, valves in accordance with this disclosure can be configured to join and mix multiple streams of multiple fluids into a single, mixed fluid flow.

The PF that can be produced by valves in accordance with this disclosure may have a number of benefits. The pulsivity of the flow may produce certain advantageous flow characteristics, such as advantageous boundary layer effects. Because of the oscillatory behavior of the boundary layer, particle carrying and surface clearing properties may be enhanced. Valves in accordance with this disclosure may therefore be a good fit for industrial scrubbers and may also be used for buildup prevention and active removal of contaminants on surfaces, for example in heat exchangers, pipes and fuel systems.

Flow control with economical control components, fast switching and high accuracy can be beneficial in industries that handle chemicals, including pharmaceuticals and petroleum. Hermetic examples of valves in accordance with this disclosure with precision manufactured valve apertures of appropriate shapes and sizes may be particularly suitable for these applications.

Steam and water flow control in power plants is another application, Additionally, example valves may also see use in heavy duty flow control regulation in Civil Engineering works like hydraulic dams, cisterns, channels, canals and levees, as well as for concrete flow control during construction.

Active control metering is another application of the disclosed rotary valves, Example valves can be used to achieve flow control with high accuracy, fast switching and with large ranges of flow rates. A valve controller can be connected in a feedback loop and pegged to some characteristic of the system, the value of which determines the system response.

Accurate flow metering, fast switching and on/off valve control for hydraulic oil or air lines may be another useful applications for rotary valves in accordance with this disclosure. Such characteristics of example valves can improve response time and increase positioning and sensing accuracy for hydraulic and pneumatic machinery, which may find applications in precision robotics and automation industry.

Another possible application for rotary valves in accordance with this disclosure is controlled power distribution from a single power source to an array or combination of independent hydraulic systems. A rapidly switching valve, in combination with hydraulic/pneumatic accumulators, can switch power between systems, running at the same time and using a single power source.

The heating, ventilation and air conditioning (HVAC) and refrigeration industries may also benefit from use of rotary valves in accordance with this disclosure. HVAC and refrigeration systems for buildings, ships, aircraft and ground vehicles need coolant and air flow control, as well as splitting and mixing of hot and cold air and/or other fluid streams, some or all of which may be achieved employing example rotary valves as disclosed and claimed herein.

Rotary valves in accordance with this disclosure may also enhance boundary layer mass transport, mixing and membrane transport with pressure waves, which effects have applications in biomedical engineering. One example is an artificial lung, where an example rotary valve may enhance mass transport and mass exchange between blood and gas and make the device more compact and more efficacious.

Another example of an application where example rotary valve may be used is in inkjet printing. In such an application natural pulsation associated with using the rotary valve may be beneficial to induced pulsation. A related application where valves in accordance with this disclosure may be used is in 3D printing or other rapid manufacturing technologies.

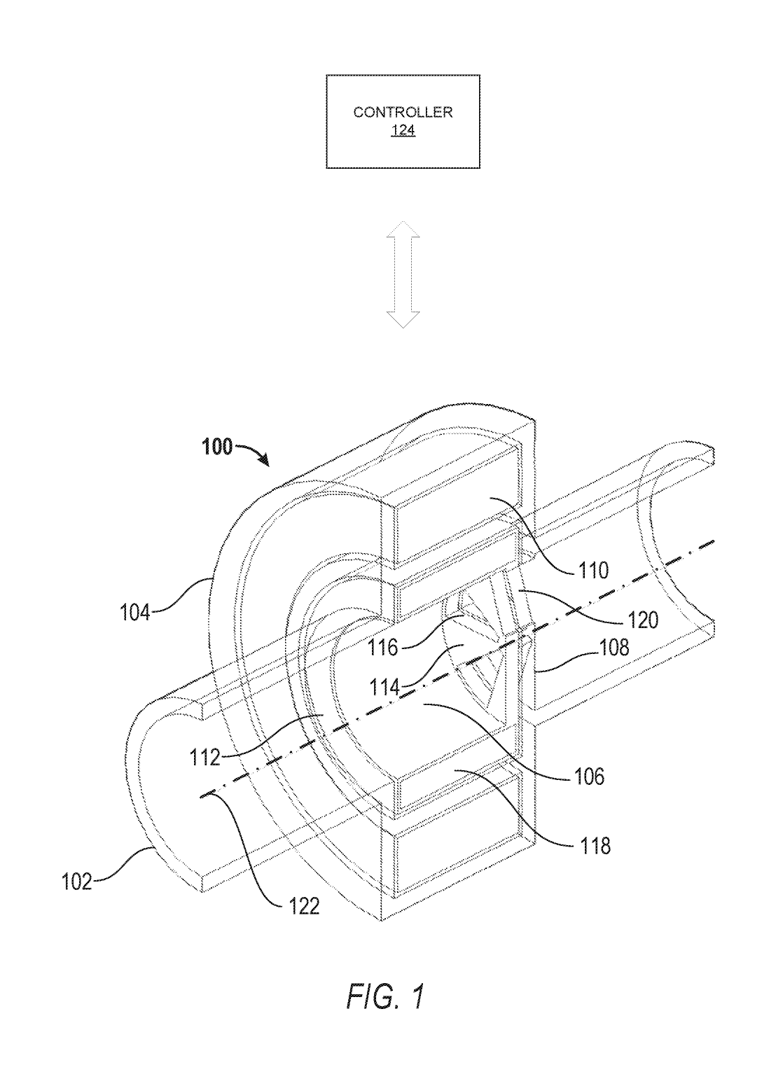

FIG. 1 depicts an example rotary valve 100 in accordance with this disclosure. In FIG. 1, valve 100 is operatively coupled to fluid conduit 102. Valve 100 includes housing 104, rotor 106, stator 108, and armature 110. Housing 104 holds rotor 106 and armature 110. Stator 108 is coupled to or integral with housing 104. In another example, stator 108 can be coupled to or integral with conduit 102. In some examples, housing 104 and/or rotary valve 100 may include seal mechanisms, including being hermetically sealed. Forming rotary valve 100 (or another example valve in accordance with this disclosure) as a hermetically sealed device may be particular advantageous or required for certain applications, including, e.g., biomedical applications.

Housing 104 is cylindrical and sized and shaped to receive armature 110 and rotor 106, which is nested within armature 110 inside housing 104. Housing 104 can be coupled to conduit 102 using various coupling mechanisms and/or fasteners. Additionally, a valve housing and fluid conduit can be integral with one another.

Rotor 106 is an annular, in this example cylindrical tubular member with open end 112 and partially closed end 114. Partially closed end 114 of rotor 106 is formed as a disc including a number of apertures 116. In other examples, rotor 106 can include more or fewer apertures 116. For example, rotor 106 can include one aperture or more than four apertures 116. In the example of FIG. 1, apertures 116 in rotor 106 are triangular or pie shaped. However, in other examples, rotor 106 can include differently shaped apertures or other openings such as slots, notches, cutouts, as examples.

Rotor 106 also includes magnetic core 118. Magnetic core 118 can be a separate component coupled to rotor 106 or can be formed integral therewith. Additionally, rotor 106 can be formed in whole or in part of a magnetic material that functions as magnetic core 118. In one example, magnetic core 118 is a permanent magnet.

Stator 108 is formed as a disc including a number of apertures 120. In other examples, stator 108 can include more or fewer apertures 120. For example, stator 108 can include one aperture or more than four apertures 120. In the example of FIG. 1, apertures 120 in stator 108 are triangular or pie shaped. However, in other examples, stator 108 can include differently shaped apertures or other openings such as slots, notches, cutouts, as examples.

Rotor 106 and stator 108 are disposed adjacent one another within valve 100 and conduit 102. Rotor 106 is configured to rotate about axis 122, which is, in this example, also the longitudinal axis of conduit 102. Stator 108 is configured to remain stationary and is aligned centrally with axis 122.

Rotary valve 100 is configured to be actively actuated by armature 110. For example, Armature 110 can include an electrical conductive member configured to convey electrical energy from a power source. Armature 110 is disposed around rotor 106, which includes magnetic core 118. By running a constant or variable electrical current through armature 110, rotor 106 can be rotated continuously or periodically, in one or two directions, to position the rotor in various positions relative to stator 106 at various constant and/or variable velocities.

For example, rotor 106 can be actuated by armature 110 to rotate at a constant angular velocity. The angular velocity of rotor 106 can be selected to produce a target frequency pulsatile flow through conduit 102, given the number and size of apertures 116 and 120 in rotor 106 and stator 108, respectively. As apertures 116 in rotor 106 rotate into alignment with apertures 120 in stator 108, fluid can flow through valve 100 and conduit 102. As apertures 116 in rotor 106 rotate out of alignment with apertures 120 in stator 108, fluid flow through valve 100 and conduit 102 is stopped.

In another example, rotor 106 can be actuated by armature 110 to rotate at a variable angular velocity. For example, rotor 106 can be actuated to rotate at a first angular velocity when apertures 116 in rotor 106 are partially or completely aligned with apertures 120 in stator 106 and at a second, different angular velocity when apertures 116 in rotor 106 are partially or completely unaligned with apertures 120.

In another example, rotor 106 can be actuated by armature 110 to rotate to a particular position relative to stator 108 at a first time and can be actuated by armature 110 to rotate into a second position relative to stator 108 at a second time. The position of rotor 106 relative to stator 108, and, in particular, the position of apertures 116 relative to apertures 120 can effectively change the cross-section of conduit 102, thereby changing the mass volume flow rate through valve 100. In this manner, rotary valve 100 may be employed to control mass/volume flow rate by varying the flow cross section of fluid conduit 102.

Although rotary valve 100 employs electrical/electromagnetic actuation mechanism, other types of actuators can be employed to actuate rotary valves in accordance with this disclosure, including, for example, mechanical, electro-mechanical, hydraulic and/or pneumatic actuators. For example, rotor 106 of rotary valve 100 can be actuated by a mechanical gear mechanism. The rim or outer periphery of rotor 106 can include gear teeth circumferentially disposed thereabout. A drive gear (or multiple gears) can be arranged to engage rotor 106 to actuate the rotor and cause it to rotate at a constant or variable angular velocity.

Actuation and operation of rotary valve 100 (and other example rotary valves in accordance with this disclosure) can be controlled in a variety of ways. In FIG. 1, rotary valve and armature 110 thereof are communicatively connected to controller 124. Controller 124 can include hardware, software, and combinations thereof to implement the functions attributed to the controller herein. Controller 124 can be an analog, digital, or combination analog and digital controller including a number of components. As examples, controller 124 can include ICB(s), PCB(s), processor(s), data storage devices, switches, relays, etcetera. Examples of processors can include any one or more of a microprocessor, a controller, a digital signal processor (DSP), an application specific integrated circuit (ASIC), a field-programmable gate array (FPGA), or equivalent discrete or integrated logic circuitry.

Storage devices, in some examples, are described as a computer-readable storage medium. In some examples, storage devices include a temporary memory, meaning that a primary purpose of one or more storage devices is not long-term storage. Storage devices are, in some examples, described as a volatile memory, meaning that storage devices do not maintain stored contents when the computer is turned off. Examples of volatile memories include random access memories (RAM), dynamic random access memories (DRAM), static random access memories (SRAM), and other forms of volatile memories known in the art. The data storage devices can be used to store program instructions for execution by processor(s) of controller 124. The storage devices, for example, are used by software, applications, algorithms, as examples, running on and/or executed by controller 124. The storage devices can include short-term and/or long-term memory, and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories.

Controller 124 can be configured to communicate with and/or directly control a power source (and associated circuitry) providing power to armature 110 of rotary valve 100. Controller 124 can be configured to communicate via various wired or wireless communications technologies and components using various public and/or proprietary standards and/or protocols. For example, a power and/or communications network of some kind may be employed to facilitate communication and control between controller 124 and rotary valve 100 (or a larger system in which the valve is employed). In one example, controller 124 may communicate via a private or public local area network (LAN), which can include wired and/or wireless elements functioning in accordance with one or more standards and/or via one or more transport mediums. In one example, controller 124 can be configured to use wireless communications according to one of the 802.11 or Bluetooth specification sets, or another standard or proprietary wireless communication protocol. Data transmitted to and from controller 124, can be formatted in accordance with a variety of different communications protocols. For example, all or a portion of the communications can be via a packet-based, Internet Protocol (IP) network that communicates data in Transmission Control Protocol/Internet Protocol (TCP/IP) packets, over, for example, Category 5, Ethernet cables or over an 802.11 or Bluetooth wireless connection.

Controller 124 can include one or more programs, circuits, algorithms or other mechanisms for controlling the operation of rotary valve 100. For example, controller 124 can be configured to modulate the speed of rotary valve 100 to produce a target PF frequency through the valve. In accordance with relatively simple programming or configuration of controller 124, rotary valve 100 can produce pressure and/or velocity fluid pulses according to a wide variety of mathematical functions.

Rotary valve 100 and other example valves in accordance with this disclosure can be fabricated using a variety of manufacturing and production methods and techniques. Additionally, valve 100 (and other example valves) can be formed of a variety of materials depending, at least in part, on the intended application. As examples, rotary valve 100 and components thereof can be fabricated from various plastics, metals, and/or ceramics. In some applications, valve 100 may be formed of non-corrosive and/or biocompatible materials.

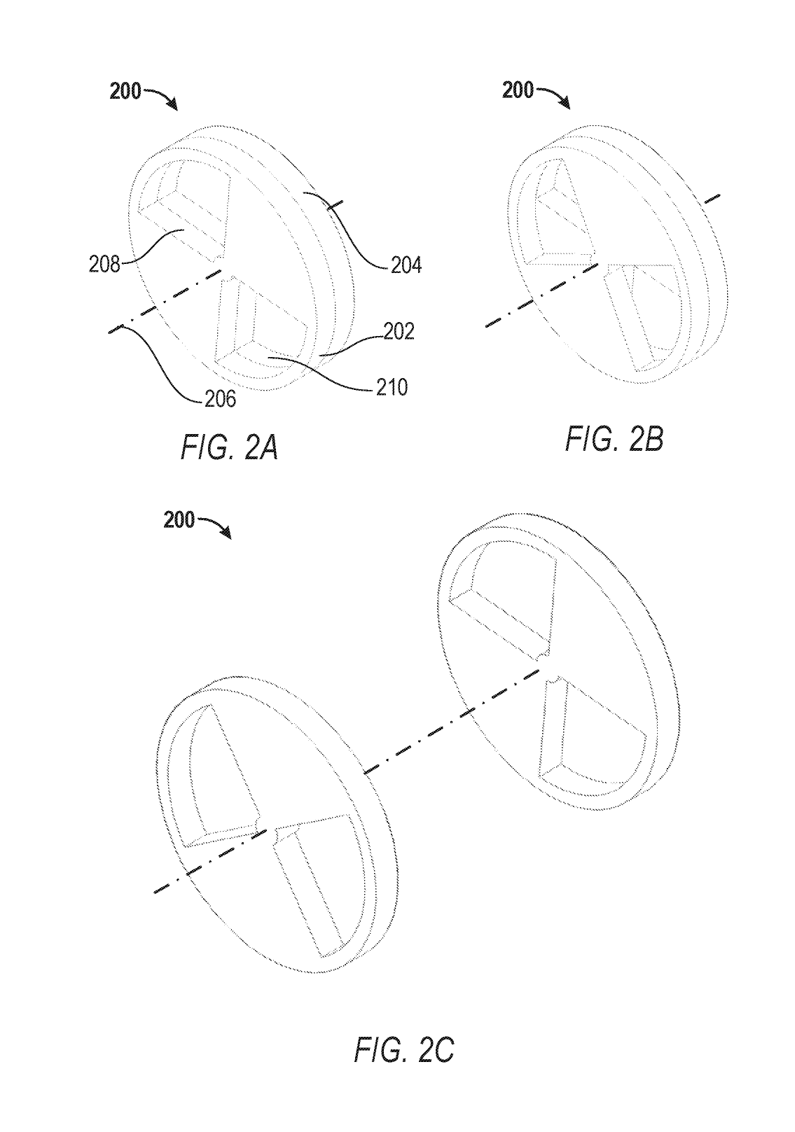

FIGS. 2A-2C depict another example valve 200 in accordance with this disclosure. Valve 200 is depicted in a simplified manner without being arranged within a particular fluid conduit or without depicting a particular actuation mechanism. However, valve 200 and other example valves described below can be disposed with respect to a conduit, actuated, and controlled in a manner consistent with that described above with reference to the example of valve 100 of FIG. 1 with the appropriate modifications for the particular example valve and intended application thereof. In the case of valve 200, for example, the valve can be arranged within or along a fluid conduit with a flow passage approximately sized to the outer diameter of valve 100 and can be actuated by one or more electrical armatures or other actuation mechanisms.

In FIGS. 2A-2C, rotary valve 200 includes two parallel discs, 202 and 204, at least one of which is rotatable about axis 206. As explained, discs 202 and 204 are configured to be operatively coupled to and arranged at least partially within a fluid conduit. Disc 202 includes two apertures 208. Disc 204 includes two apertures 210. Apertures 208 and 210 in discs 202 and 204, respectively, are triangular or pie shaped. However, in other examples, discs 202 and 204 can include differently shaped apertures or other openings such as slots, notches, cutouts, as examples. Additionally, each of discs 202 and 204 can include more or fewer than two apertures. By controlling the shape of apertures 208 and 210 and the angular velocity of one or both of discs 202 and 204 it is possible to attain fluid flows that fit virtually any desired velocity or pressure field. In some cases, both discs 202 and 204 rotate. In another example, one of discs 202 and 204 is a rotor and the other a stator.

Valve 200 can be actuated to rotate one or both of discs 202 and 204 at a constant or variable angular velocity, and one or both of discs 202 and 204 in one or multiple directions. The angular velocity of the rotating valve members of valve 200, whether one or both of discs 202 and 204, can be selected to produce a target frequency pulsatile flow through a conduit. As apertures 208 in disc 202 rotate into alignment with apertures 210 in disc 204, fluid can flow through valve 200 and a fluid conduit to which the valve is operatively coupled. As apertures 208 in disc 202 rotate out of alignment with apertures 210 in disc 204, fluid flow through valve 200 is stopped.

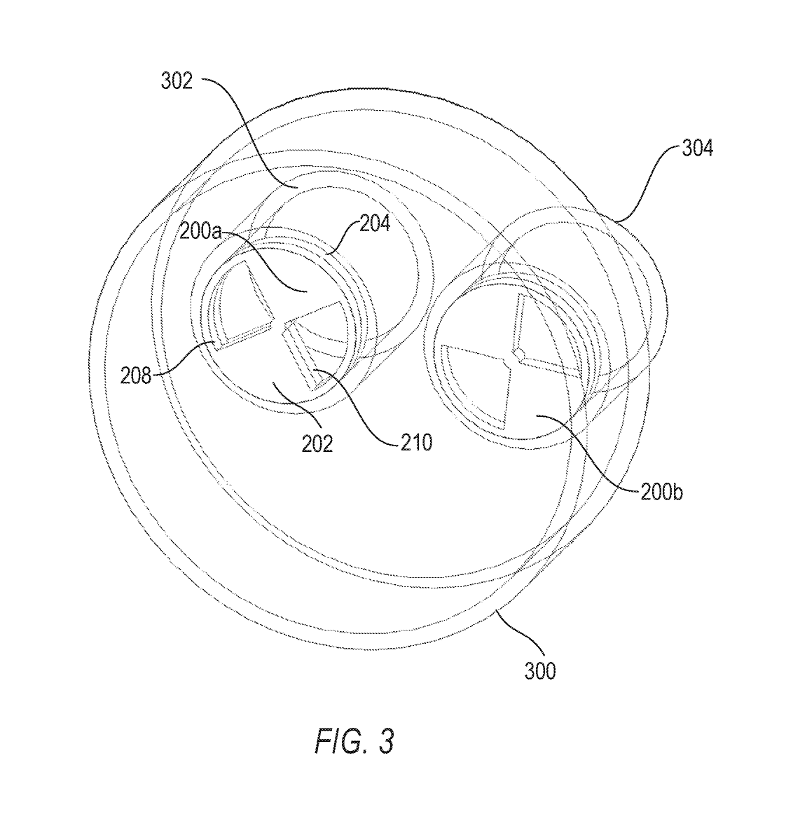

Rotary valve 200 can be employed in a variety of applications. FIG. 3, for example, depicts two rotary valves 200a and 200b employed in a flow splitting or flow mixing application (depending upon the direction of fluid flow). In FIG. 3, fluid flows on one side of valves 200 through a single conduit 300 and on the other side of valves 200 through two separated conduits 302 and 304. In other flow splitting/mixing examples, more than two conduits and two example valves may be employed.

Both of the two valves 200a and 200b are arranged in conduit 300. Valve 200a is also arranged in conduit 302 and valve 200b is arranged in conduit 304. Depending upon the direction of fluid flow, valves 200a and 200b can be employed to combine and mix two fluids flowing separately through conduits 302 and 304 into a mixed fluid flowing through conduit 300, or if flow is reversed, can be employed to direct a fluid flowing through conduit 300 into one or both of conduits 302 and 304. Although this example refers to two valves 200a and 200b within various conduits 300-304, this entire system/device can also be considered a single valve that splits one fluid into multiple channels or combines and mixes multiple fluids into one channel.

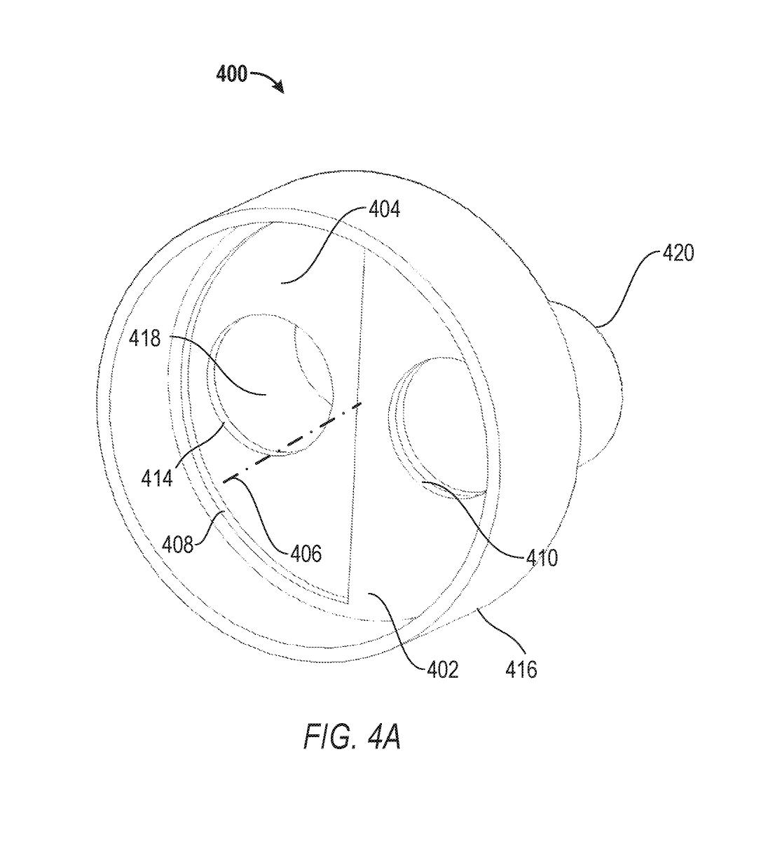

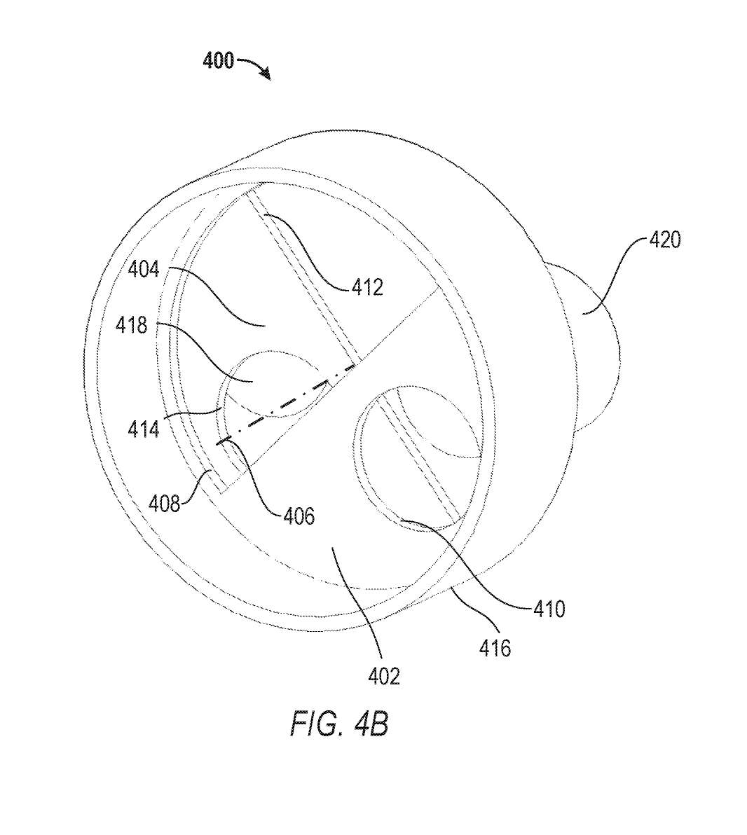

FIGS. 4A and 4B depict another example rotary valve 400 in accordance with this disclosure. In FIGS. 4A and 4B, valve 400 includes first rotor 402 and second rotor 404, both of which are configured to rotate about axis 406. Rotor 402 (formed as a disc) includes first aperture 408 and second aperture 410. Rotor 404 (also formed as a disc) includes first aperture 412 and second aperture 414. Valve 400 is operatively coupled to conduits 416, 418 and 420 to, depending upon the direction of fluid flow, split flow from conduit 416 into conduits 418 and 420, or, if fluid flow is reversed, to combine and mix flows from conduits 418 and 420 in conduit 416.

As with some other example rotary valves in accordance with this disclosure, rotor 402 and rotor 404 (or in other examples, rotor and stator, two discs or two valve members) may be identical to one another. For example, each of rotor 402 and 404 includes first aperture 408 and 412, which is semi-circular, and second aperture 410 and 414, which is circular. Additionally, the size and orientations of the first and second apertures 408-412 may be the same for both rotor 402 and 404. The identity of these parts in example valves in accordance with this disclosure can decrease the complexity and cost of production.

Valve 400 can be actuated to rotate rotors 402 and 404 at a constant or variable angular velocity, and in the same or different directions, and each rotor in one or two directions. The angular velocity of rotors 402 and 404 of valve 400 can be selected to produce a target frequency pulsatile flow through a conduit, including through conduit 416 or through conduits 418 and 420. As apertures 408 and 410 in rotor 402 rotate into alignment with apertures 412 and 414 in rotor 404, fluid can flow through valve 400 and conduits 416 and 418 and/or 420. apertures 408 and 410 in rotor 402 rotate out of alignment with apertures 412 and 414 in rotor 404, fluid flow through valve 400 and associated conduits 416-420 is stopped.

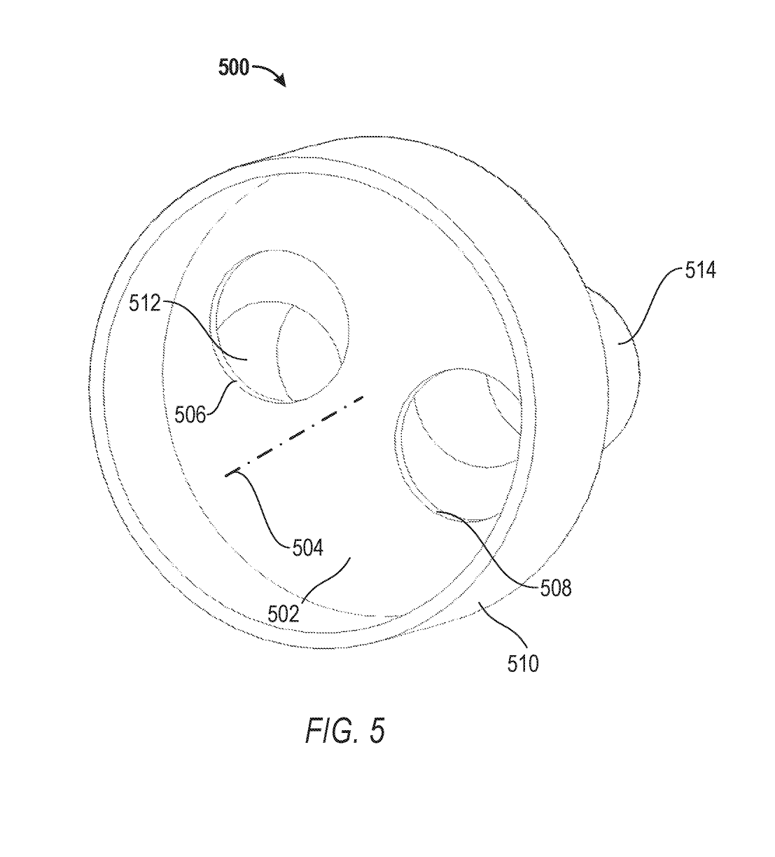

FIG. 5 depicts another example rotary valve 500. In FIG. 5, valve 500 includes a single rotor 502, which is configured to rotate about axis 504. Rotor 502 (formed as a disc) includes first aperture 506 and second aperture 508. Valve 500 is operatively coupled to conduits 510, 512 and 514 to, depending upon the direction of fluid flow, split flow from conduit 510 into conduits 512 and 514, or, if fluid flow is reversed, to combine and mix flows from conduits 512 and 514 in conduit 510.

Valve 500 can be actuated to rotate rotor 502 at a constant or variable angular velocity, and in one or multiple directions. The angular velocity of rotor 502 of valve 500 can be selected to produce a target frequency pulsatile flow through a conduit, including through conduit 510 or through conduits 512 and 514. In this example, as apertures 506 and 508 in rotor 502 rotate into alignment with conduits 512 and 514, respectively, fluid can flow through valve 500 and conduits 510 and 512 and/or 514. As apertures 506 and 508 in rotor 502 rotate out of alignment with conduits 512 and 514, fluid flow through valve 500 and associated conduits 510-514 is stopped.

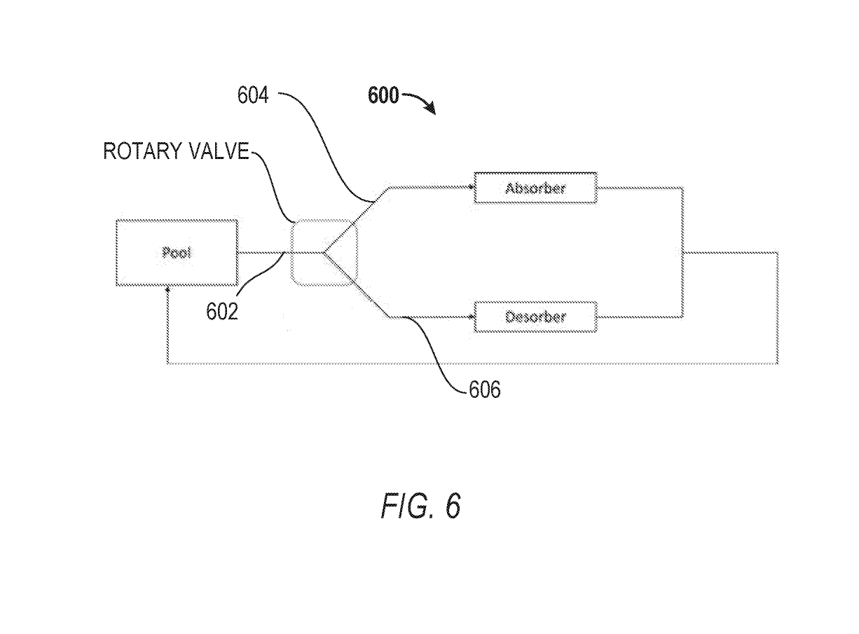

Valves in accordance with the examples of FIGS. 3-5 could be employed in a variety of particular systems. For example, FIG. 6 schematically depicts chlorine level control system 600 for a swimming pool. In FIG. 6, water exits the swimming pool through conduit 602 and engages a rotary valve (or valves, for example, valve(s) 200, 400 or 500) at a T-junction. The rotary valve can be controlled to direct varying amounts of the water through conduits 604 and 606 to the chlorine adsorber and desorber devices, respectively, to control the amount of chlorine in the pool.

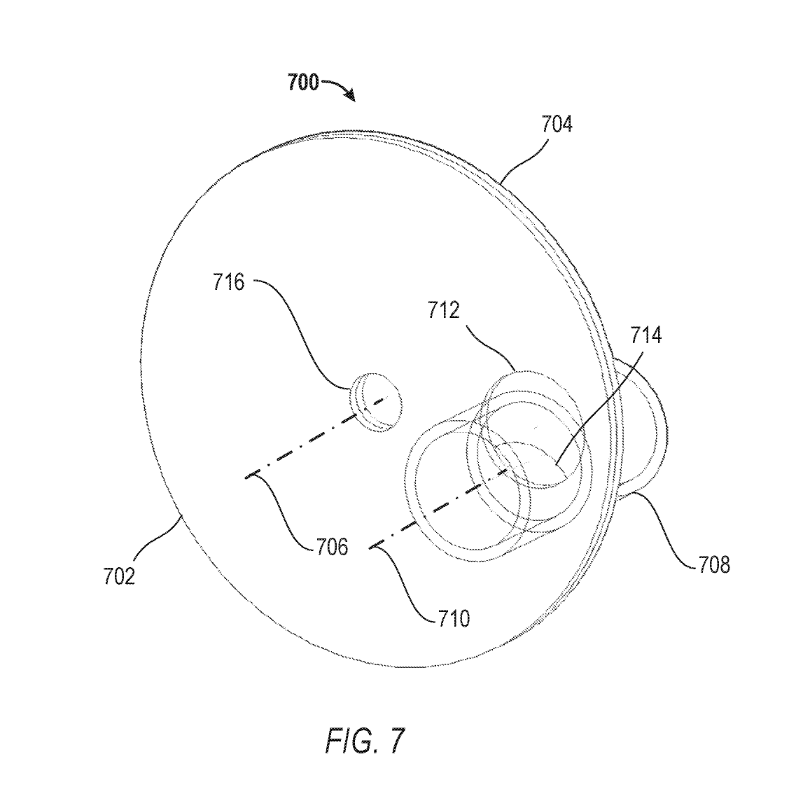

FIG. 7 depicts another example rotary valve 700 in accordance with this disclosure. In FIG. 7, valve 700 includes rotor 702 and rotor 704, which are configured to rotate about axis 706 to control fluid flow through conduit 708. Fluid conduit 708 includes longitudinal axis 710, which, in this example valve 700, is offset from the axis of rotation 706 of rotors 702 and 704. Rotor 702 includes one aperture 712 (although in other examples such a rotor can include more apertures) and rotor 704 includes one aperture 714. The size and shape of apertures 704 and 706 match the size and shape of the fluid flow passage of fluid conduit 708. In this case, when rotors 702 and 704 are rotated into a position at which apertures 712 and 714 are in full alignment with the passage of fluid conduit 708, the passage of the conduit and fluid flowing there through are substantially completely unobstructed. This feature may be advantageous in certain applications, including, e.g., biomedical applications where obstructions in fluid flow may cause cellular or other damage to the biological fluid flowing through a valve.

Valve 700 can be actuated to rotate rotors 702 and 704 at a constant or variable angular velocity, and each rotor in one or multiple directions. The angular velocity of rotors 702 and 704 of valve 700 can be selected to produce a target frequency pulsatile flow through conduit 708. As depicted in FIG. 7, each of rotors 702 and 704 includes a central thru hole 716, which is coaxial with axis of rotation 706. Although not shown, a drive or bearing shaft could be received in central holes of 716 of rotors 702 and 704 as part of an actuation mechanism therefore. As aperture 712 in rotor 702 rotates into alignment with aperture 714 of rotor 704 and conduit 708, fluid can flow through valve 700 and conduit 708. As aperture 712 in rotor 702 rotates out of alignment with aperture 714 of rotor 704 and conduit 708, fluid flow through valve 700 and conduit 708 is stopped.

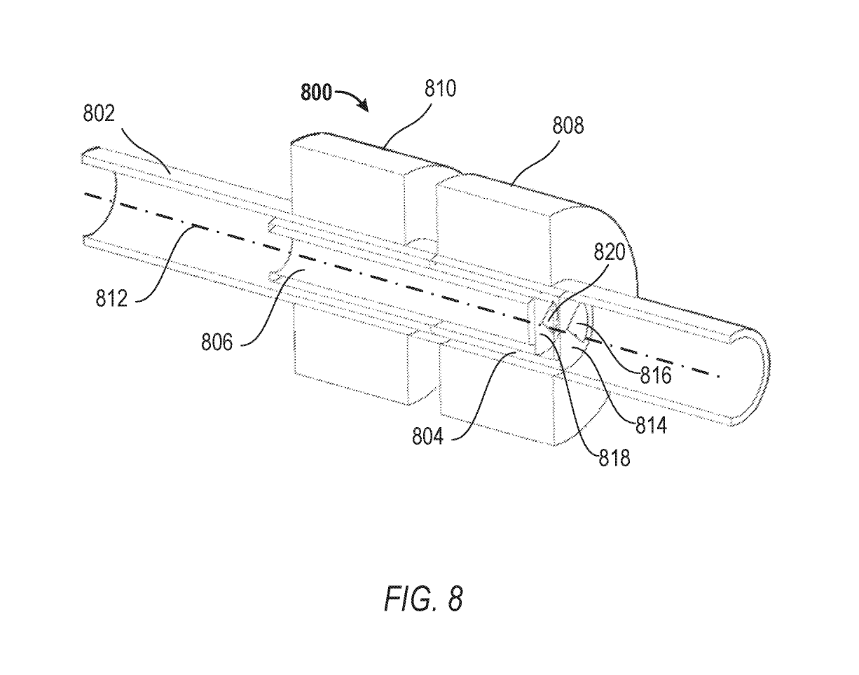

FIG. 8 is a section view depicting another example rotary valve 800 in accordance with this disclosure. In FIG. 8, valve 800 is operatively coupled to fluid conduit 802. Valve 800 includes outer rotor 804, inner rotor 806, outer rotor armature 808 and inner rotor armature 810. Valve 800 is configured to be actuated by one or both of armatures 808 and 810 to rotate one or both of rotors 804 and 806 about axis 812 (axis of rotation of rotors/valve and longitudinal axis of conduit) to control flow of a fluid through conduit 802.

Outer rotor 804 and inner rotor 806 are what may be referred to as nested tubular rotors. Outer rotor 804 is an annular, in this example cylindrical tubular member with an open end and partially closed end 814. Partially closed end 814 of rotor 804 is formed as a disc including an aperture 816. Inner rotor 806 is also an annular, in this example cylindrical tubular member with an open end and partially closed end 818. Partially closed end 818 of rotor 806 is formed as a disc including an aperture 820. In other examples, one or both of rotors 804 and 806 can include more than one aperture. In the example of FIG. 1, apertures 816 and 820 are triangular or pie shaped. However, in other examples, rotors 804 and 806 can include differently shaped apertures or other openings such as slots, notches, cutouts, as examples.

Inner rotor 806 is nested or received in outer rotor 804. FIG. 8 depicts ends 814 and 818 of outer rotor 804 and inner rotor 806 as offset, but this is at least in part to illustrate the structure of each rotor. In operation, partially ends 814 and 818 of outer rotor 804 and inner rotor 806 including apertures 816 and 820 may abut one another so that the closed end of the inner rotor is pushed up against the closed end of the outer rotor. Inner rotor 806 extends beyond and is longer than outer rotor 804 and the nested outer and inner rotors of example rotary valve 800 are disposed in and along the flow path of fluid conduit 802.

Each of rotors 804 and 806 include a magnetic core are formed at least in part from a magnetic material. Magnetic cores of rotors 804 and 806 can be a separate component coupled to each rotor or can be formed integral therewith.

Rotary valve 800 is configured to be actively actuated by one or both of armatures 808 and 810 being energized to rotate one or both of outer rotor 804 and inner rotor 806. As with other example valves in accordance with this disclosure, each of rotors 804 and 806 can be controllably actuated to rotate in one or two directions and at a constant or variable angular velocity. Additionally, each of rotors 804 and 806 can rotate at the same speed in the same directions or at different speeds in different directions, or at the same speeds in different directions or at different speeds in the same direction.

The angular velocity of rotors 804 and 806 can be selected to produce a target frequency pulsatile flow through conduit 802, given the number and size of apertures 816 and 820. As aperture 816 in rotor 804 rotates into alignment with aperture 820 in rotor 806, fluid can flow through valve 800 and conduit 802. As aperture 816 in rotor 804 rotates out of alignment with aperture 820 in rotor 806, fluid flow through valve 800 and conduit 802 is stopped.

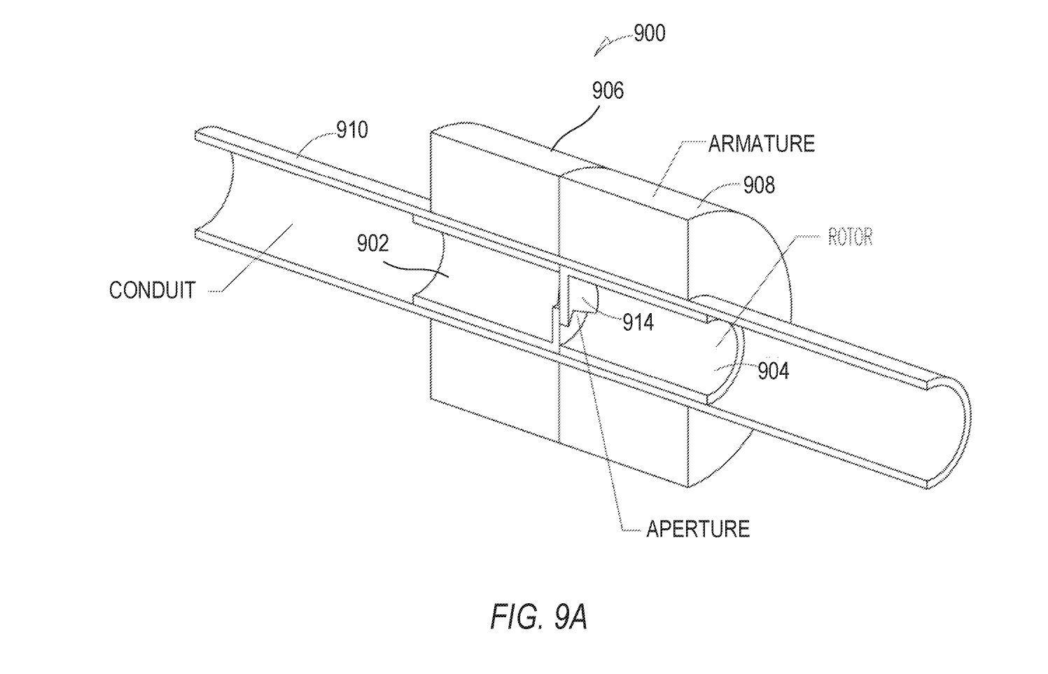

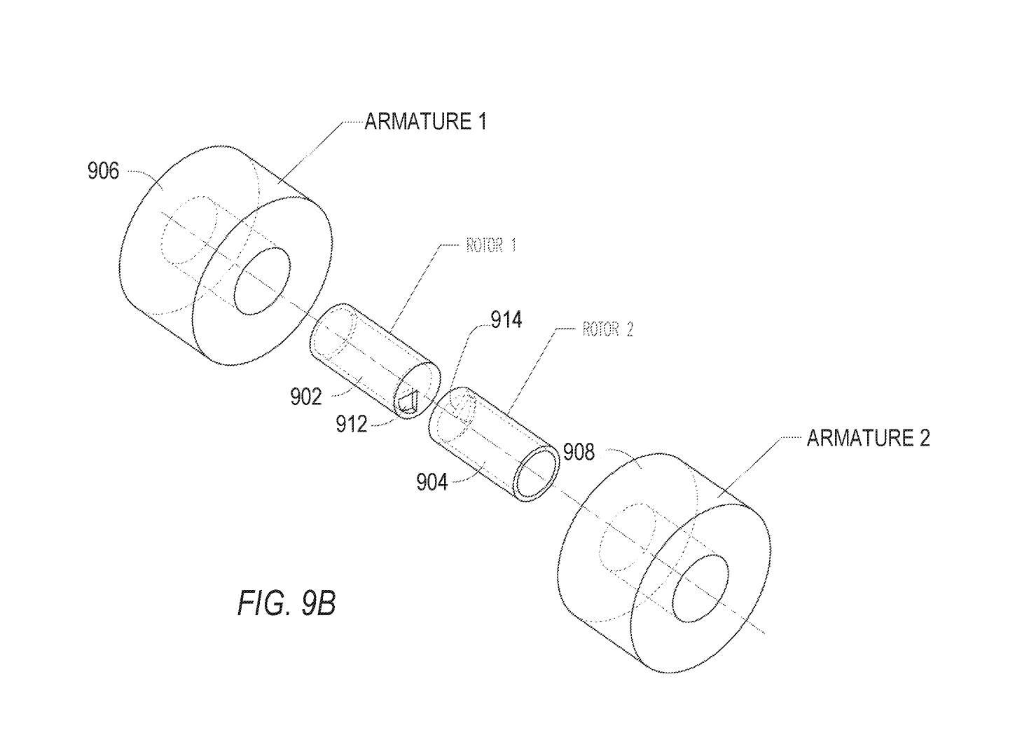

FIGS. 9A and 9B depict another example valve 900 in accordance with this disclosure. Rotary valve 900 is very similar in structure and function to rotary valve 800, except that instead of nested tubular outer and inner rotors 804 and 806 as with valve 800, valve 900 includes end-on tubular rotors 902 and 904 actuated by armatures 906 and 908 to rotate the rotors to control the flow of fluid through conduit 910. Rotor 902 includes aperture 912 and rotor 904 includes aperture 914. In other examples, rotors 902 and 904 could include more than one aperture each, including two, like in the example of valve 200, or four like the example of the valve 100.

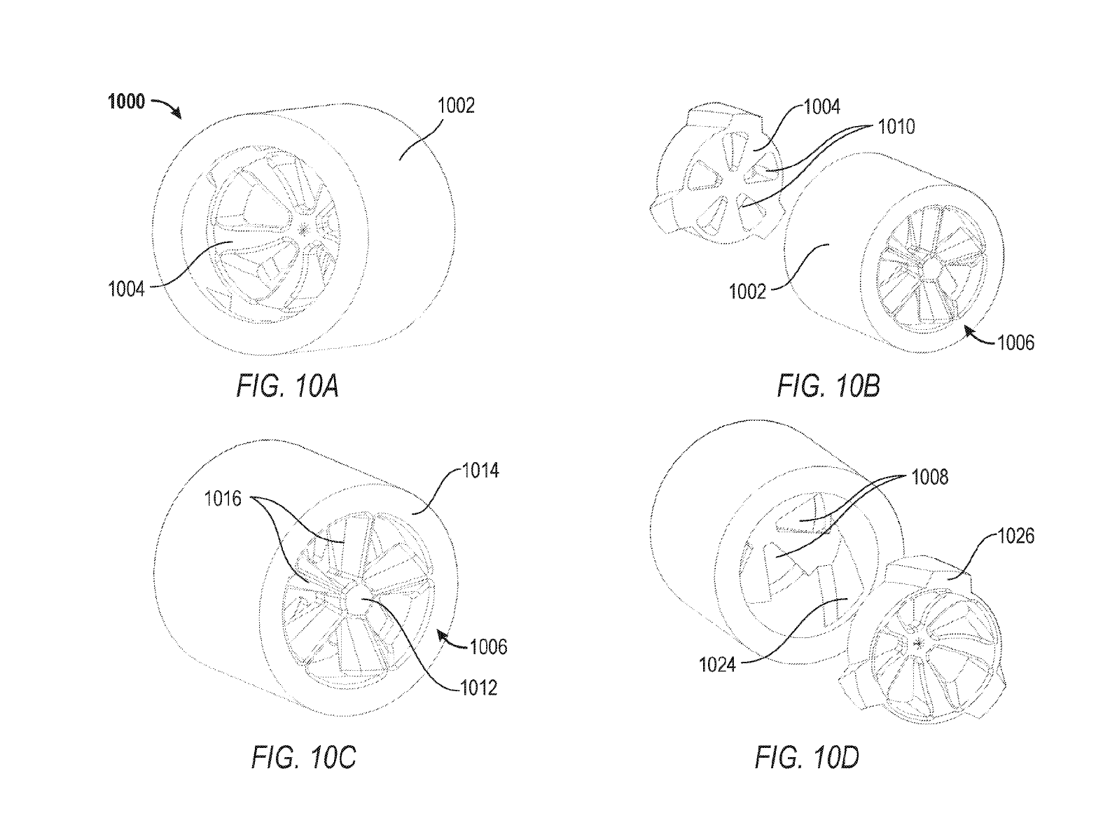

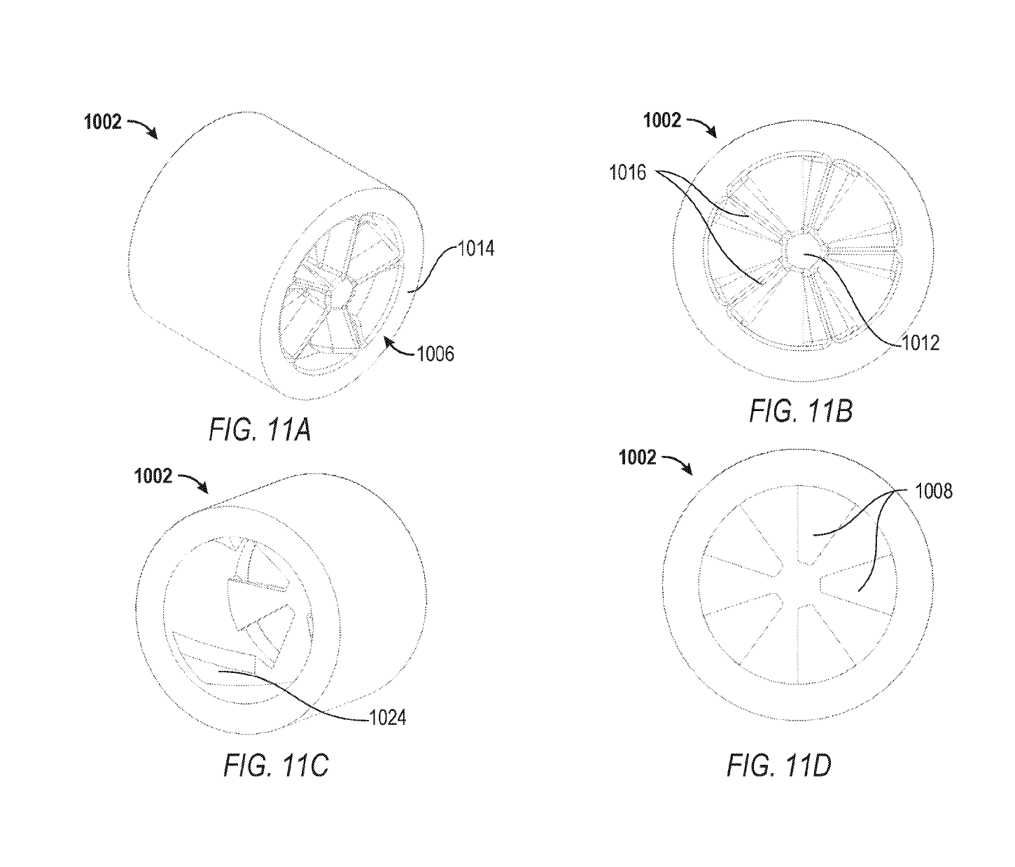

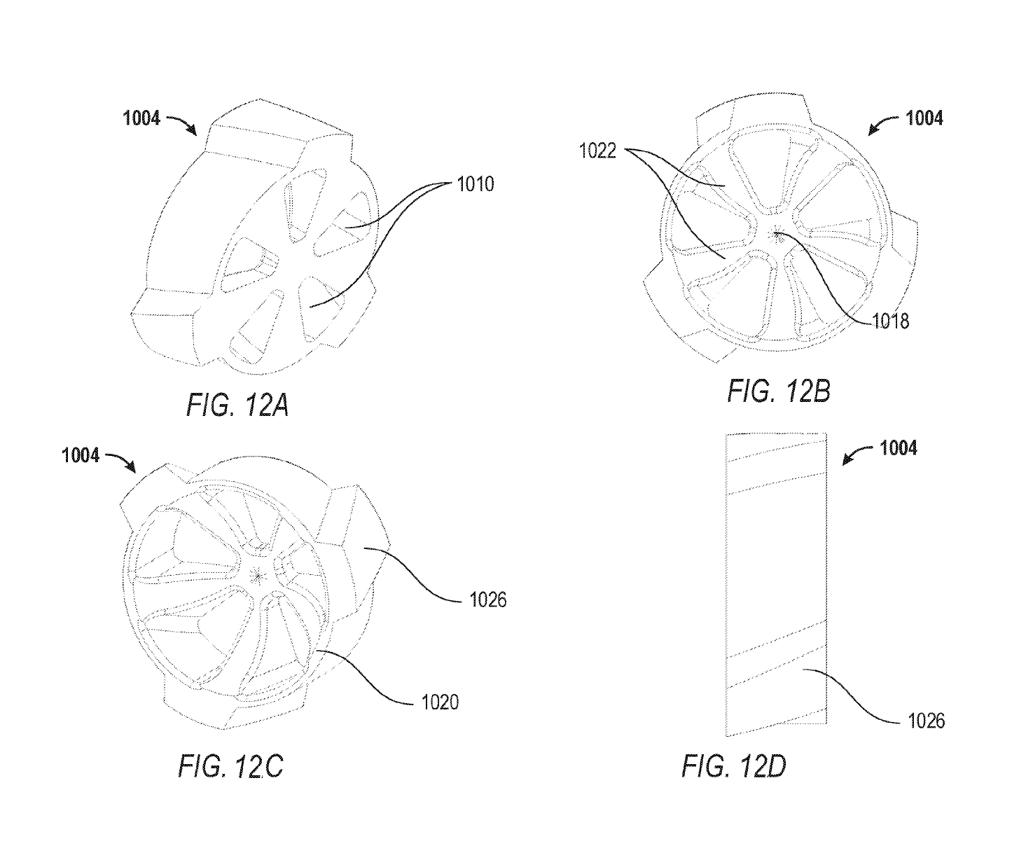

FIGS. 10A-10D, 11A-11D, and 12A-12D depict another example rotary valve 1000 in accordance with this disclosure. FIGS. 10A-10D depict valve 1000 assembled and exploded in a number of views. FIGS. 11A-11D depict stator 1002 of valve 1000 and FIGS. 12A-121) depict rotor 1004 of valve 1000.

As with other example rotary valves described herein, although not specifically depicted for described below, valve 1000 can be disposed with respect to a conduit, actuated, and controlled in a manner consistent with that described above with reference to the example of valve 100 of FIG. 1, with the appropriate modifications for valve 1000 and the intended application thereof. In the case of valve 1000, for example, the valve can be arranged within or along a fluid conduit with a flow passage approximately sized to the diameter of valve 1000 and can be actuated by one or more electrical armatures or other actuation mechanisms and controlled by a hardware, software, or hardware and software based electronic control device or system.

Rotary valve 1000 includes stator 1002 and rotor 1004. Rotor 1004 is disposed within and configured to rotate relative to stator 1002. Rotor 1004 is also configured to translate axially relative to stator 1002, as described in more detail below.

Stator 1002 is an annular, in this example cylindrical tubular member with an open end and partially closed end 1006. Partially closed end 1006 of stator 1002 can be described as formed as a disc, as with other examples described above, including a number of apertures 1008. Similarly, rotor 1004 can be described as formed as a disc including a number of apertures 1010. The overall structure and configuration of partially closed end 1006 of stator 1002 and stator 1004 is, as evident from FIGS. 10A-12D, similar to an automobile wheel rim. For example, partially closed end 1006 includes hub 1012 aligned with a center of the end, rim 1014 defining a circumference or outer periphery, and a number of spokes 1016, each of which extend between the hub and the rim. Each of apertures 1008 is defined by a space between adjacent spokes 1016 and between hub 1012 and rim 1014 of partially closed end 1006 of stator 1002.

Rotor 1004 includes hub 1018 aligned with the center of the rotor, rim 1020 defining a circumference or outer periphery, and a number of spokes 1022, each of which extend between the hub and the rim. Each of apertures 1010 of rotor 1004 is defined by a space between adjacent spokes 1022 and between hub 1018 and rim 1020 of rotor 1004. This hub, rim and spoke structure/nomenclature of valve 1000 is also applicable to the rotors and stators of example valves 100, 200, 800 and 900.

Stator 1002 also includes a number of grooves 1021 in the inner surface thereof. In the example of FIGS. 10A-12D, stator 1002 includes three grooves, but, in other examples, the stator could include more or fewer grooves. Grooves 1024 extend axially along the inner surface of stator 1002 along a curved path. In some cases, grooves 1024 can extend axially along the inner surface of stator 1002 along a helical path. Rotor 1004 includes a number of flanges 1026, which extend radially outward from the periphery (for example, outward from rim 1020) of the rotor. Additionally, as best seen in FIGS. 12A-12D, flanges 1026 have a curved, e.g., helical axial profile from one end of rotor 1004 to the opposite end.

Rotary valve 1000 can be actuated to rotate rotor 1004 relative to stator 1002 in a variety of ways. Regardless of the actuation mechanism, however, rotary valve 1000 is configured to be actuated to cause rotor 1004 to rotate and translate axially into multiple positions. For example, rotary valve 1000 is configured to be actuated to cause rotor 1004 to rotate and translate axially into a closed position in which rotor 1004 is adjacent and abutting partially closed end 1006 of stator 1002 and spokes 1016 of stator 1002 align with apertures 1010 of rotor 1004 to substantially stop fluid flow through the rotary valve. Additionally, rotary valve 1000 is configured to be actuated to cause rotor 1004 to rotate and translate axially into an open position in which rotor 1004 is axially offset from partially closed end 1006 of stator 1002 and apertures 1008 of stator 1002 are at least partially aligned with apertures 1010 of rotor 1004 to allow fluid flow through rotary valve 1000.

In one example, rotary valve 1000 is passively actuated. For example, rotary valve 1000 can be arranged at least partially within and operatively connected to a fluid conduit. Rotary Rotor 1004 can be actuated to rotate and translate axially relative to and within stator 1002 by a pressure differential on either side of valve 1000.

In another example, rotary valve 1000 is actively actuated. For example, electrically conductive coils or an electrically conductive material can be embedded in, coupled to or formed integral with spokes 1016 of stator 1002 and a magnetic material can be embedded in, coupled to or formed integral with spokes 1022 of rotor 1004. The coils in spokes 1016 of stator 1002 can be coupled to a power source to selectively and controllably drive current through the coils (e.g., using a controller like controller 150 as described with reference to the example of FIG. 1) to actuate valve 1000 to cause rotor 1004 to rotate and translate axially relative to and within stator 1002 to open and close the valve.

In an example, stator 1002 could include two, opposite partially closed ends, each of which includes a hub, rim and spoke structure similar to that described above with reference to partially closed end of stator 1002. As with other examples described above (and equally applicable as appropriate to examples described below), directionality (polarity) and the amount of current in the electric coils of spokes 1016 of stator 1002 will affect the force of attraction/repulsion between the electric coil and magnetic spokes 1022 of rotor 1004. The electric coils of stationary spokes 1016 can thereby be used to actuate rotor 1004, which allows for position and velocity control of rotor 1004.

In order to improve and/or modulate fluid flow characteristics across rotary valve 1000, spokes 1016 of stator 1002 and spokes 1022 of rotor 1004 are formed, at least in part, as airfoils. Forming spokes (or other solid structures in the path of fluid flow) as airfoils or other contoured shapes can improve pressure, velocity and other characteristics (e.g., boundary layer conditions) of fluid flow across rotary valves in accordance with this disclosure. Improving and/or modulating fluid flow characteristics using the structure of the valve may be advantageous in a number of different applications. For example, in medical applications the imposition of a valve including one or more solid structures in the flow path may cause blood flowing there through to be damaged (e.g., at a cellular level) or have some other untoward effect. Thus, forming spokes 1016 of stator 1002 and spokes 1022 of rotor 1004 are formed, at least in part, as airfoils may diminish such effects. Additionally, spokes 1016 and/or spokes 1022 of valve 1000 may be designed/optimized to target some beneficial flow characteristic, including reducing pressure loss, boundary layer flow, or some other beneficial characteristic of the fluid flow as it passes through the valve.

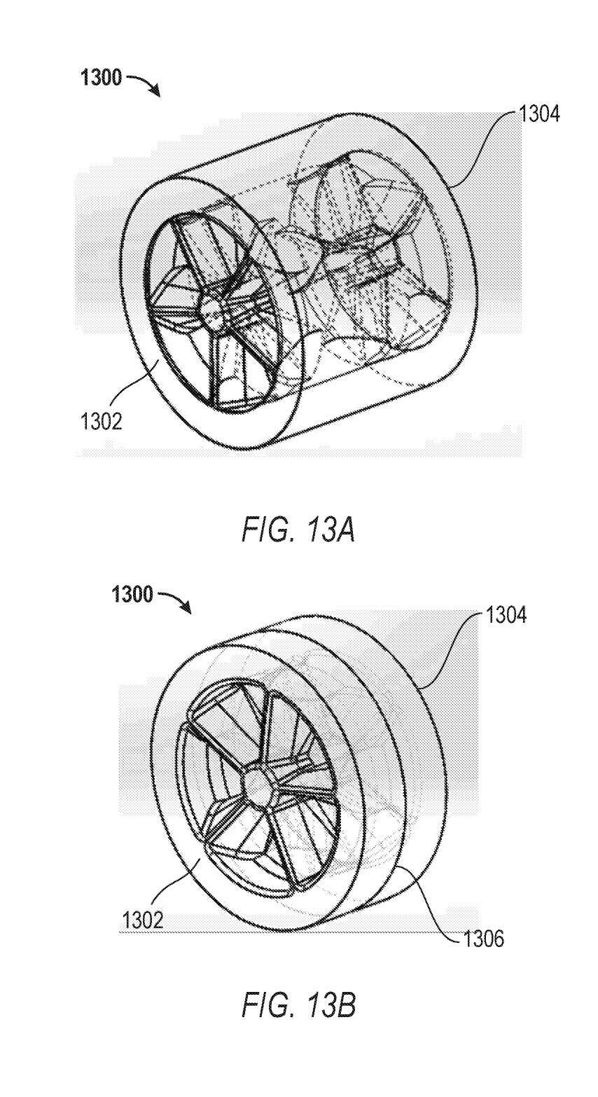

FIGS. 13A and 13B depict another example valve 1300 in accordance with this disclosure. Rotary valve 1300 is similar to rotary valve 1000 except that valve 1300 includes two, opposite partially closed ends 1302 and 1304 and flexible wall 1306 there between. Each of ends 1302 and 1304 of valve 1300 includes a structure similar to or the same as partially closed end 1006 of stator 1002 and rotor 1004 of valve 1000, including a hub, rim, and spoke structure as described above. In some cases, one of ends 1302 and 1304 can be considered a rotor and the other of ends 1302 and 1304 can be considered a stator. In other cases, both of ends 1302 and 1304 can be considered rotors.

Flexible wall 1306 is configured to be actuated to cause one or both of partially closed ends to rotate and translate axially from the open position depicted in FIG. 13A to the closed state depicted in FIG. 13B. Flexible wall 1306 could be constructed in a number of ways and include a number of components. For example, flexible wall 1306 could be constructed of a flexible outer and inner shell and a number of shape memory members there between, which can be selectively actuated to cause the outer and inner shells to collapse and/or rotate and thereby cause relative rotation and translation of ends 1302 and 1304 to cause valve 1300 to open and close. The shape memory members could be formed form a variety of materials, including, for example a Nickel-Titanium allow such as Nitinol.

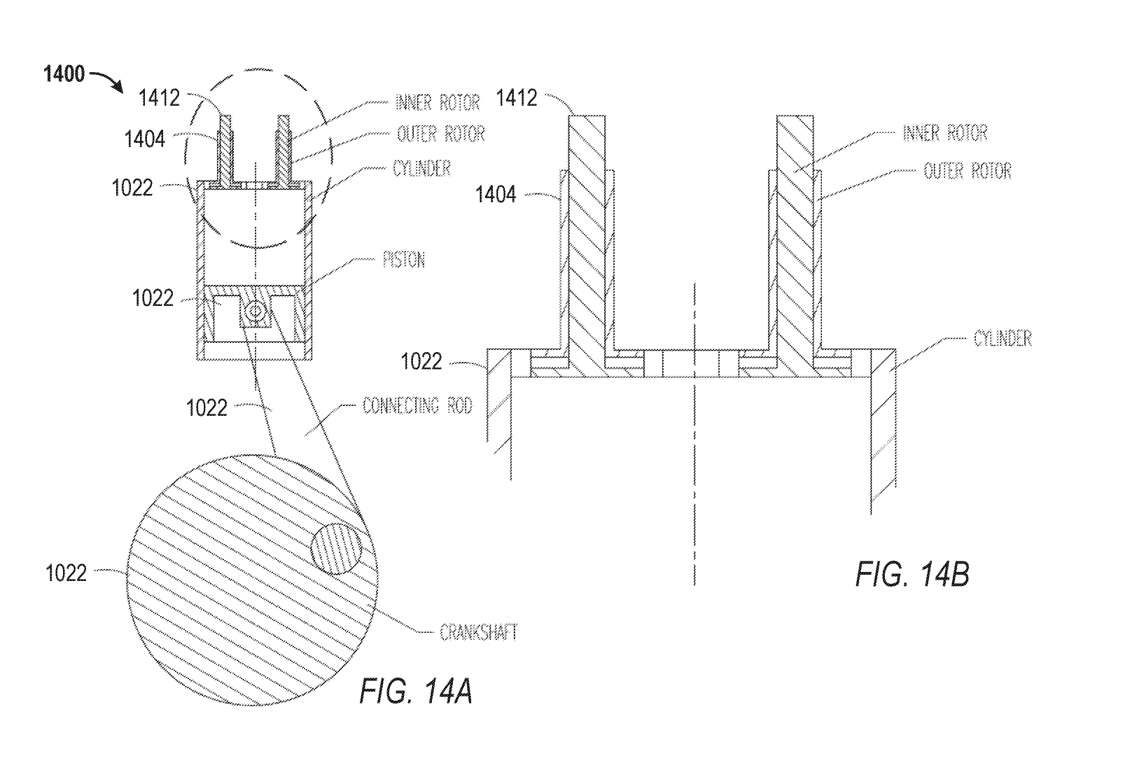

In some cases, rotary valves in accordance with this disclosure can be employed in an internal combustion (IC) piston engine used for automotive or power generation purposes, which uses valves to control fluidic movement into and out of a cylinder. FIGS. 14A and 14B schematically depict IC engine 1400 including two rotary valves 1402 in accordance with this disclosure. It is possible to actuate several of rotary valves 1402 using a timing chain at the same time, whereas in at least some existing VVT designs each valve typically requires its own solenoid.

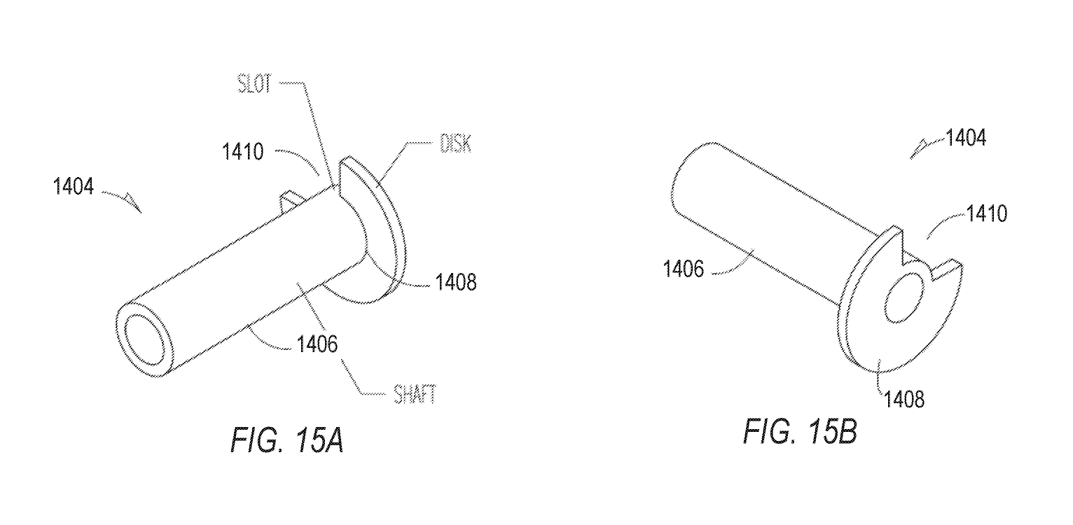

FIGS. 15A and 158 depict outer rotor 1404 of valve 1402 in more detail. Outer rotor 1404 can be a rotatable member, including a hollow (or annular) shaft (or stem) 1406. Rotor 1402 also includes disc 1408 with a slot (or cutout) 1410 extending radially outward from annular shaft 1406.

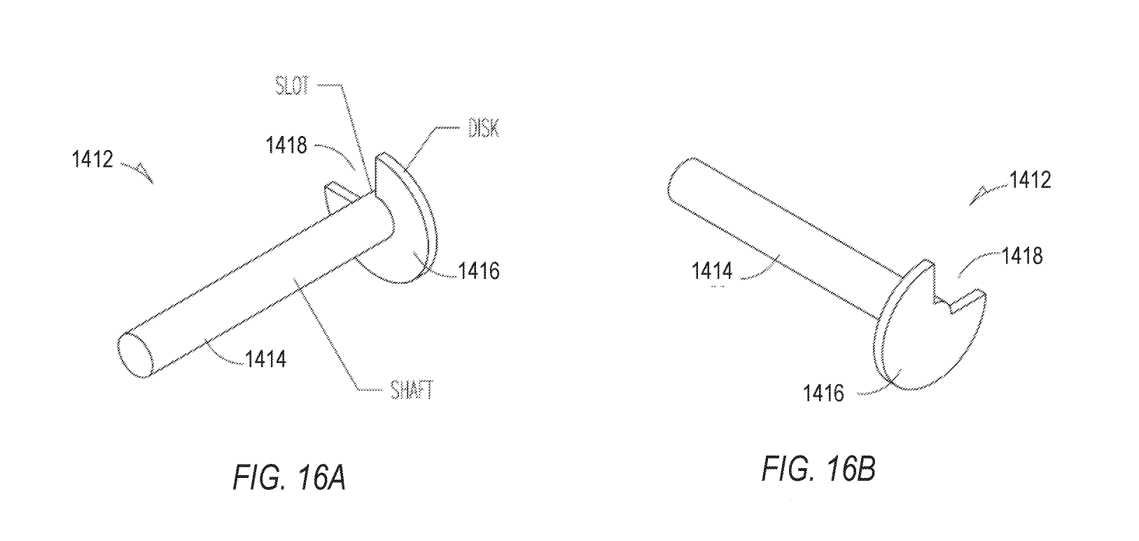

FIGS. 16A and 16B depict inner rotor 1412 of valve 1402 in more detail. Inner rotor 1412 can also be a rotatable member with shaft (or stem) 1414 with disc 1416 extending radially outward therefrom. Disc 1416 includes slot (or cutout) 1418.

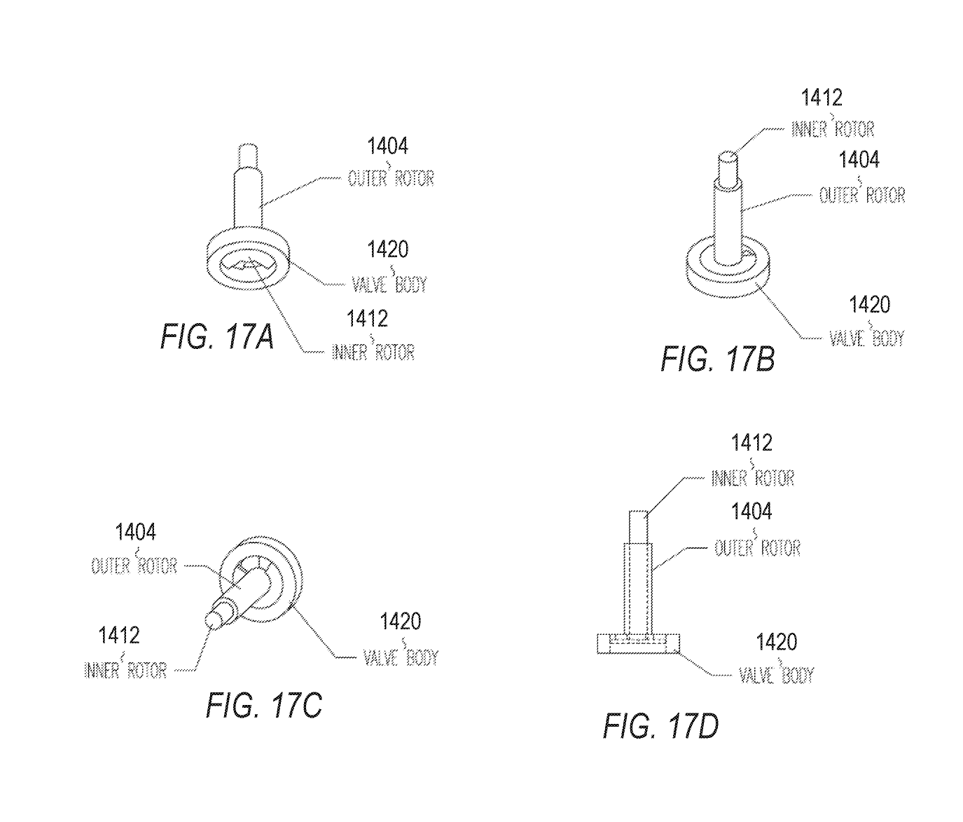

FIGS. 17A-17D depict rotary valve 1402 including valve body 1420, inner rotor 1412 and outer rotor 1404. Although valve 1402 includes valve body 1402 as a separate component, this structure could, in other examples, be incorporated into/integral with the device that receives and employs valve 1402 like the cylinder IC engine 1400.

As shown in FIGS. 17A-17D, the solid shaft of inner rotor 1412 is received in hollow shaft 1406 of outer rotor 1404 and at least one of discs 1408 and 1416 are housed within valve body 1420. In an open position, slot 1410 of disc 1408 is aligned with slot 1418 in disc 1416 to permit fluidic flow. In a closed position slot 1410 of disc 1408 is positioned relative to slot 1418 in disc 1416 to block fluid flow. Rotary valve 1402 may also be controlled to modulate mass volume flow rate by varying the flow cross section, thus any number of flow patterns may be established including flow patterns defined with desired functions.

In operation, a valve with an inner rotor 22 and an outer rotor 20 allows gas to enter a cylinder 1450 which upon combustion exerts pressure on a piston 1452 causing movement of a connecting rod 1454 to turn a crankshaft 1456. Several of these assemblies may be housed in a cylinder-head of a piston IC engine (shown as a “flathead” type piston IC engine here). Some of these assemblies can act as valves for the intake manifold of the engine, from which fresh air (or air+fuel) will be aspirated at appropriate times, and some of these assemblies will act as valves for the exhaust manifold of the engine, which will expel exhaust at appropriate times.

Thus, as shown in FIGS. 14A-17D, an example rotary valve in accordance with this disclosure includes a solid shaft, at one end of which a slotted disc is mounted, and which is housed in a hollow shaft, at whose end a slotted disc is mounted as well. These two make for an “inner rotor” and an “outer rotor”, respectively. The inner and outer rotors are housed in a valve body. These rotors are constrained from any motion except rotation about their shaft's central axis. When the slots in the discs overlap, the mechanism can transmit a fluidic flow, which is otherwise blocked. By selecting the shape and size of the slots for a specific application, and by varying the speed and direction of rotation of the rotors, a required frequency and duration of valve opening can be set for that particular application. Thus, VVT for a piston IC engine may be achieved in this manner.

The size and configuration of the slots or openings can be used to control flow patterning. In the case where several rotary valves are actuated, for example, by a timing chain, the size and configuration of the slots or openings can be used to control the frequency and duration of valve opening, based on a particular application. In one form, the slots or openings may be configured as wedge cutouts. Alternatively, the slots or openings may comprise a plurality of different holes of the same or different sizes. Of course, the slots associated with one disc need not be the same as the other disc. The size and geometry of the slots or openings may be designed to achieve particular effects.

Another benefit of the valve is that rapid flow rates may be achieved. In addition, pulsation may be induced in order to produce desired effects. For example, pulsation may assist in mixing. This pulsation may be achieved through controlling how the valve rotates.

It should also be understood that fluidic activity and parameters upstream of the valve may also be adjusted in order to obtain desired effects from the valve on fluidic flow. Similarly, operational parameters of the valve, such as RPM, duration of opening and frequency of opening can be adjusted to alter fluidic parameters upstream of the valve. For example, the size and shape of the cavity upstream of the valve may be engineered in order to maximize desired effects of oscillation. For example, the size and geometry of a chamber located upstream of one or more valves may be designed to provide a resonance cavity. It is noted that the effects of this valve within a system may readily be modeled using commercially available simulation programs and other known design and manufacturing tools. Thus, the ability to produce pulsation and oscillation may be leveraged as a benefit instead of attempting to avoid such effects altogether. For example, in the context of an injection system, this oscillation may be used to assist in eliminating leakage and provide an extra boost in pressure.

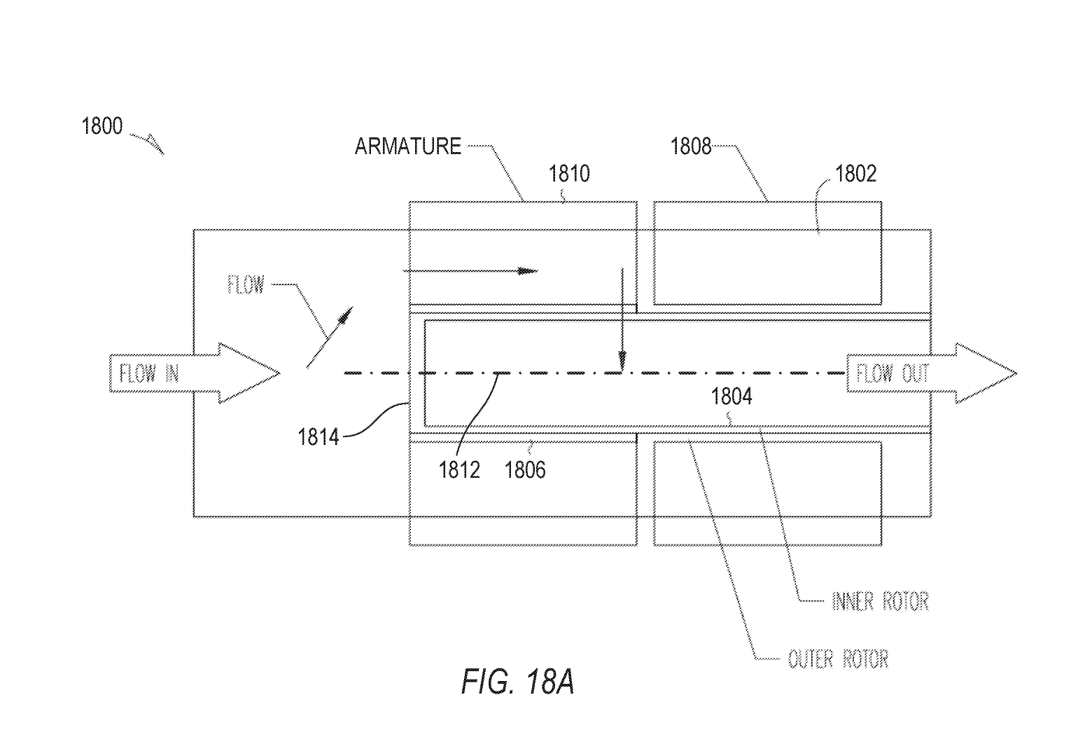

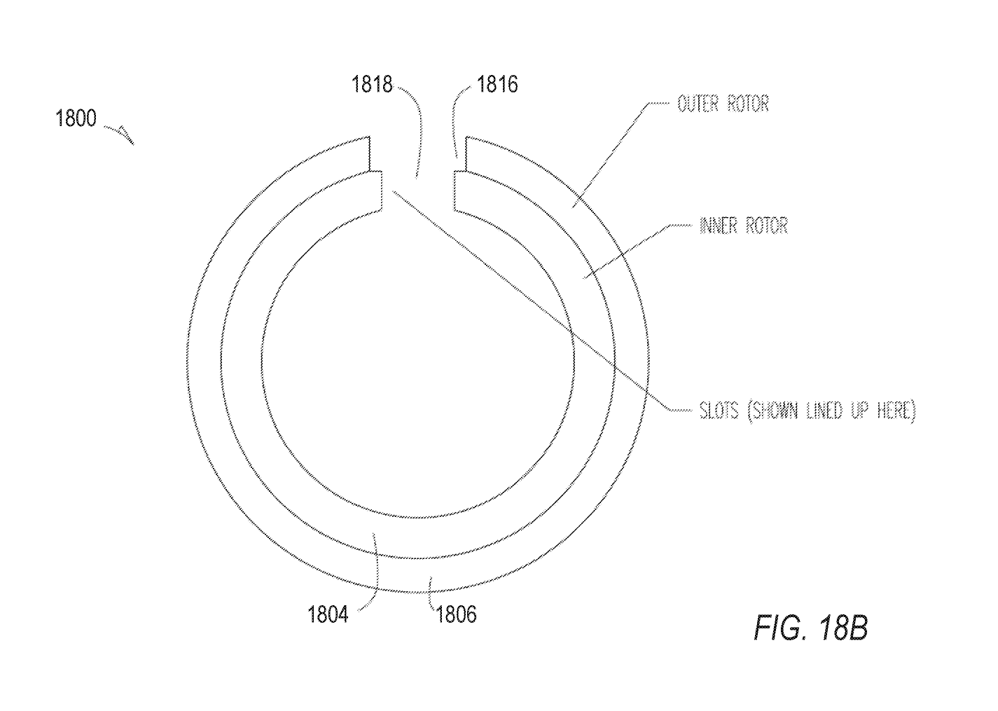

FIG. 18A depicts another example rotary valve 1800 operatively coupled and configured to control fluid flow through conduit 1802. FIG. 18B depicts inner rotor 1804 and outer rotor 1806 of rotary valve 1800. In the example of FIGS. 18A and 188, rotary valve 1800 includes inner rotor 1804, outer rotor 1806, inner rotor armature 1808, and outer rotor armature 1810. Valve 1800 is configured to be actuated by one or both of armatures 1808 and 1810 to rotate one or both of rotors 1804 and 1806 about axis 1812 (axis of rotation of rotors/valve and longitudinal axis of conduit) to control flow of a fluid through conduit 1802.

Outer rotor 1804 and inner rotor 1806 are what may be referred to as nested tubular rotors. Outer rotor 1806 is an annular, in this example cylindrical tubular member with an open end and completely closed end 1814. Outer rotor 1804 also includes aperture, in this example a longitudinal slot 1816 in the outer circumference of the rotor. Inner rotor 1804 is also an annular, in this example cylindrical tubular member with two open ends. Inner rotor 1804 includes an aperture, in this example a longitudinal slot 1818 in the outer circumference of the rotor. In other examples, inner rotor 1804 and outer rotor 1806 could include multiple apertures, for example, multiple slots. Slots 1816 and 1818 in outer rotor 1804 and inner rotor 1806 can be straight or linear, or, in other examples, the longitudinal slots can have a curved profile, including, e.g., a hyperbolic, parabolic and elliptic.

Inner rotor 1804 is nested or received in outer rotor 1806. Inner rotor 1804 extends beyond and is longer than outer rotor 1806 and the nested outer and inner rotors of example rotary valve 1800 are disposed in and along the flow path of fluid conduit 1802.

Each of rotors 1804 and 1806 include a magnetic core or are formed at least in part from a magnetic material. Magnetic cores of rotors 1804 and 1806 can be a separate component coupled to each rotor or can be formed integral therewith.

Rotary valve 1800 is configured to be actively actuated by one or both of armatures 1808 and 1810 being energized to rotate one or both of inner rotor 1804 and outer rotor 1806. As with other example valves in accordance with this disclosure, each of rotors 1804 and 1806 can be controllably actuated to rotate in one or two directions and at a constant or variable angular velocity. Additionally, each of rotors 1804 and 1806 can rotate at the same speed in the same directions or at different speeds in different directions, or at the same speeds in different directions or at different speeds in the same direction.

The angular velocity of rotors 1804 and 1806 can be selected to produce a target frequency pulsatile flow through conduit 1802, given the number and size of apertures 1816 and 1818. As aperture 1816 in rotor 1806 rotates into alignment with aperture 1818 in rotor 1804, fluid can flow through valve 1800 and conduit 1802. As aperture 1816 in rotor 1806 rotates out of alignment with aperture 1818 in rotor 1804, fluid flow through valve 1800 and conduit 1802 is stopped.

### NOTES & EXAMPLES

The present application provides for the following exemplary embodiments or examples, the numbering of which is not to be construed as designating levels of importance:
Example 1 provides a rotary valve comprising: a first valve member configured to be disposed at least partially within a fluid conduit and comprising at least one first aperture; a second valve member configured to be disposed at least partially within the fluid conduit and comprising at least one second aperture, and at least one of the first and second valve members being rotatable to be positioned in a plurality of positions relative to one another, and a position of the at least one first aperture relative to the at least one second aperture controlling fluid flow through the rotary valve.
Example 2 provides the rotary valve of Example 1 and optionally wherein the at least one of the first and second valve members being rotatable is configured to rotate periodically.
Example 3 provides the rotary valve of Example 1 and optionally wherein the at least one of the first and second valve members being rotatable is configured to rotate continuously.
Example 4 provides the rotary valve of Example 1 and optionally wherein the at least one of the first and second valve members being rotatable is configured to rotate in one or more directions.
Example 5 provides the rotary valve of Example 1 and optionally wherein the at least one of the first and second valve members being rotatable is configured to be rotated to be positioned in one or more first positions in which the at least one first aperture and the at least one second aperture are at least partially aligned to allow fluid to flow through the rotary valve and a second position in which the at least one first aperture and the at least one second apertures are unaligned to substantially stop fluid flow through the rotary valve.
Example 6 provides the rotary valve of Example 1 and optionally wherein the at least one of the first and second valve members being rotatable is configured to be rotated to be positioned in a first position in which the at least one first aperture and the at least one second aperture are at least partially aligned at a first time and a second position in which the at least one first aperture and the at least one second aperture are at least partially aligned at a first time to vary the volumetric or mass flow rate through the rotary valve.
Example 7 provides the rotary valve of Example 1 and optionally wherein: the first valve member comprises a stator; and the second valve member comprises a rotor; and the second valve member is rotatable relative to the first valve member.
Example 8 provides the rotary valve of Example 7 and optionally wherein the stator comprises an annulus comprising an open end and a second end comprising a first disc and the rotor comprises a second disc disposed and configured to rotate within the annulus adjacent the first disc, the first disc comprising the at least one first aperture and the second disc comprising the at least one second aperture.
Example 9 provides the rotary valve of Example 7 and optionally wherein the rotor comprises an annulus comprising an open end and a second end comprising a first disc and the stator comprises a second disc disposed adjacent the first disc, the first disc comprising the at least one first aperture and the second disc comprising the at least one second aperture.
Example 10 provides the rotary valve of Examples 8-9 and optionally wherein each of the first and second discs comprise: a hub aligned with a center of the disc; a rim defining a circumference of the disc; and a plurality of spokes, each of which extend between the hub and the rim, wherein the at least one first aperture comprises a plurality of first apertures, each of which is defined by a space between adjacent spokes and between the hub and the rim of the first disc, and wherein the at least one second aperture comprises a plurality of second apertures, and wherein a space between adjacent spokes and between the hub and the rim of the second disc defines one of the plurality of first apertures.
Example 11 provides the rotary valve of Example 10 and optionally wherein the annulus comprises at least one groove on an inner surface thereof, and wherein the second disc comprises at least one flange extending radially outward from a periphery of the second disc, the at least one flange received within the at least one groove.
Example 12 provides the rotary valve of Example 11 and optionally wherein the at least one groove extends axially from adjacent the first disc along a curved path.
Example 13 provides the rotary valve of Example 11 and optionally wherein the at least one groove extends axially from adjacent the first disc along a helical path.
Example 14 provides the rotary valve of Example 11 and optionally wherein the second disc is configured to rotate and translate axially relative to the first disc.
Example 15 provides the rotary valve of Example 13 and optionally wherein the second disc is configured to rotate and translate axially relative to the first disc into a closed position in which the second disc is adjacent the first disc and the plurality of spokes of the second disc align with the plurality of first apertures of the first disc to substantially stop fluid flow through the rotary valve and cause the second disc to rotate and translate axially into an open position in which the second disc is axially offset from the first disc and the plurality of second apertures of the second disc are at least partially aligned with the plurality of first apertures of the first disc to allow fluid flow through the rotary valve.
Example 16 provides the rotary valve of Example 1 and optionally wherein: the first valve member comprises a first rotor; and the second valve member comprises a second rotor; and the first and second valve members are rotatable relative to one another.
Example 17 provides the rotary valve of Example 16 and optionally wherein: the first rotor comprises an annulus comprising an open end and a second end comprising a first disc, the first disc comprising the at least one first aperture; and the second rotor comprises an annulus comprising an open end and a second end comprising a second disc, the second disc comprising the at least one second aperture.
Example 18 provides the rotary valve of Example 17 and optionally wherein the second rotor is arranged within the annulus of the first rotor, the second disc being arranged adjacent the first disc.
Example 19 provides the rotary valve of Example 17 and optionally wherein the first and second rotors are disposed in an end-on arrangement with the first and second discs facing and adjacent one another and the first and second rotors axially aligned.
Example 20 provides the rotary valve of Example 16 and optionally wherein: the first rotor comprises a first disc comprising the at least one first aperture; and the second rotor comprises a second disc comprising the at least one second aperture.
Example 21 provides the rotary valve of Example 20 and optionally wherein: the first disc comprises two apertures arranged approximately opposite one another about a center of the first disc; and the second disc comprises two apertures arranged approximately opposite one another about a center of the second disc.
Example 22 provides the rotary valve of Example 21 and optionally wherein: a first aperture of the two apertures of the first disc comprises a semi-circle and a second aperture of the two apertures of the first disc comprises a circle.
Example 23 provides the rotary valve of Example 21 and optionally wherein: a first aperture of the two apertures of the second disc comprises a semi-circle and a second aperture of the two apertures of the second disc comprises a circle.
Example 24 provides the rotary valve of Example 20 and optionally wherein each of the first and second discs comprises one aperture.
Example 25 provides the device of Example 24 and optionally wherein the one aperture of each of the first and second discs is approximately the same size as the fluid conduit.
Example 26 provides the rotary valve of Example 25 and optionally wherein an axis of rotation of the first and second discs is configured to be offset from a longitudinal axis of the fluid conduit.
Example 27 provides the rotary valve of Example 18 and optionally wherein: the first rotor comprises an annulus comprising two open ends; the at least one first aperture comprises a longitudinal slot in a circumference of the first rotor; the second rotor comprises an annulus comprising an open end and a closed end; and the at least one second aperture comprises a longitudinal slot in a circumference of the second rotor.
Example 28 provides the rotary valve of Examples 17 and 19-27 and optionally wherein the first rotor is identical to the second rotor.
Example 29 provides the rotary valve of Example 1 and optionally wherein: the first valve member comprises a stem, one end of which includes an at least partially circular first disc extending radially outward from the stem, the first disc comprising the at least one aperture; the second valve member comprises an annular stem, one end of which includes an at least partially circular second disc extending radially outward from the annular stem, the second disc comprising at least one aperture; and the stem of the first valve member is received within the annular stem of the second valve member such that the first disc is adjacent the second disc.
Example 30 provides the rotary valve of Examples 1-27 and optionally wherein the structure of the first valve member is identical to the structure of the second valve member.
Example 31 provides the rotary valve of Examples 1-30 and optionally wherein the valve further comprises an actuator, the actuator configured to rotate at least one of the first and second valve members to cause the valve members to be positioned in the plurality of positions relative to one another to control flow of the fluid through the body.
Example 32 provides the rotary valve of Example 31 and optionally wherein the actuator comprises a mechanical, electrical, electro-mechanical, or electromagnetic actuator.
Example 33 provides the rotary valve of Example 31 and optionally wherein the actuator comprises: at least one electrical armature connected to a power source and configured to receive a constant or variable electrical current therefrom; and at least one magnetic member coupled to or integral with at least one of the first and second valve members, the at least one electrical armature surrounding the at least one magnetic member.
Example 34 provides the rotary valve of Examples 1-30 and optionally wherein the valve is a passive valve, wherein a pressure differential between first and second sides of the rotary valve cause at least one of the first and second valve members to rotate to be positioned in a plurality of positions relative to one another.
Example 35 provides a rotary valve comprising: at least one rotatable valve member configured to be operatively connected to and rotate relative to a fluid conduit, the at least one rotatable valve member comprising at least one aperture, and the at least one rotatable valve member being rotatable to be positioned in a plurality of positions, a position of the at least one aperture relative to the fluid conduit controlling fluid flow through the rotary valve and the fluid conduit.
Example 36 provides a method comprising: operatively coupling a rotary valve to a conduit, the rotary valve comprising at least one rotatable valve member configured to rotate relative to the conduit, the at least one rotatable valve member comprising at least one aperture; and rotating the at least one rotatable valve member into a plurality of positions to control fluid flow through the fluid conduit.

Various examples according to this disclosure have been described. These and other examples are within the scope of the following claims.

1. A rotary valve comprising:
a first valve member configured to be disposed at least partially within a fluid conduit and comprising at least one first aperture;
a second valve member configured to be disposed at least partially within the fluid conduit and comprising at least one second aperture, and
at least one of the first and second valve members being rotatable to be positioned in a plurality of positions relative to one another, and a position of the at least one first aperture relative to the at least one second aperture controlling fluid flow through the rotary valve,
wherein the stator comprises an annulus comprising an open end and a second end comprising a first disc and the rotor comprises a second disc disposed and configured to rotate within the annulus adjacent the first disc, the first disc comprising the at least one first aperture and the second disc comprising the at least one second aperture,
wherein each of the first and second discs comprise:
a hub aligned with a center of the disc;
a rim defining a circumference of the disc; and
a plurality of spokes, each of which extend between the hub and the rim,
wherein the at least one first aperture comprises a plurality of first apertures, each of which is defined by a space between adjacent spokes and between the hub and the rim of the first disc, and
wherein the at least one second aperture comprises a plurality of second apertures, and wherein a space between adjacent spokes and between the hub and the rim of the second disc defines one of the plurality of first apertures, and
wherein the annulus comprises at least one groove on an inner surface thereof, and wherein the second disc comprises at least one flange extending radially outward from a periphery of the second disc, the at least one flange received within the at least one groove.

2. The rotary valve of claim 1, wherein the at least one of the first and second valve members being rotatable is configured to rotate periodically.

3. The rotary valve of claim 1, wherein the at least one of the first and second valve members being rotatable is configured to rotate continuously.

4. The rotary valve of claim 1, wherein the at least one of the first and second valve members being rotatable is configured to rotate in one or more directions.

5. The rotary valve of claim 1, wherein the at least one of the first and second valve members being rotatable is configured to be rotated to be positioned in one or more first positions in which the at least one first aperture and the at least one second aperture are at least partially aligned to allow fluid to flow through the rotary valve and a second position in which the at least one first aperture and the at least one second apertures are unaligned to substantially stop fluid flow through the rotary valve.

6. The rotary valve of claim 1, wherein the at least one of the first and second valve members being rotatable is configured to be rotated to be positioned in a first position in which the at least one first aperture and the at least one second aperture are at least partially aligned at a first time and a second position in which the at least one first aperture and the at least one second aperture are at least partially aligned at a second time to vary the volumetric or mass flow rate through the rotary valve.

7. The rotary valve of claim 1, wherein:
the first valve member comprises a stator; and
the second valve member comprises a rotor; and
the second valve member is rotatable relative to the first valve member.

8.-11. (canceled)

12. The rotary valve of claim 1, wherein the at least one groove extends axially from adjacent the first disc along a curved path.

13. The rotary valve of claim 1, wherein the at least one groove extends axially from adjacent the first disc along a helical path.

14. The rotary valve of claim 1, wherein the second disc is configured to rotate and translate axially relative to the first disc.

15. The rotary valve of claim 14, wherein the second disc is configured to rotate and translate axially relative to the first disc into a closed position in which the second disc is adjacent the first disc and the plurality of spokes of the second disc align with the plurality of first apertures of the first disc to substantially stop fluid flow through the rotary valve and cause the second disc to rotate and translate axially into an open position in which the second disc is axially offset from the first disc and the plurality of second apertures of the second disc are at least partially aligned with the plurality of first apertures of the first disc to allow fluid flow through the rotary valve.

16. A rotary valve comprising:
a first valve member configured to be disposed at least partially within a fluid conduit and comprising at least one first aperture;
a second valve member configured to be disposed at least partially within the fluid conduit and comprising at least one second aperture, and
at least one of the first and second valve members being rotatable to be positioned in a plurality of positions relative to one another, and a position of the at least one first aperture relative to the at least one second aperture controlling fluid flow through the rotary valve, wherein:
the first valve member comprises a first rotor; and
the second valve member comprises a second rotor; and
the first and second valve members are rotatable relative to one another.

17. The rotary valve of claim 16, wherein:
the first rotor comprises an annulus comprising an open end and a second end comprising a first disc, the first disc comprising the at least one first aperture; and
the second rotor comprises an annulus comprising an open end and a second end comprising a second disc, the second disc comprising the at least one second aperture.

18. The rotary valve of claim 17, wherein the second rotor is arranged within the annulus of the first rotor, the second disc being arranged adjacent the first disc.

19. The rotary valve of claim 17, wherein the first and second rotors are disposed in an end-on arrangement with the first and second discs facing and adjacent one another and the first and second rotors axially aligned.

20. The rotary valve of claim 16, wherein:
the first rotor comprises a first disc comprising the at least one first aperture; and
the second rotor comprises a second disc comprising the at least one second aperture.

21. The rotary valve of claim 20, wherein:
the first disc comprises two apertures arranged approximately opposite one another about a center of the first disc; and
the second disc comprises two apertures arranged approximately opposite one another about a center of the second disc.

22. The rotary valve of claim 21, wherein:
a first aperture of the two apertures of the first disc comprises a semi-circle and a second aperture of the two apertures of the first disc comprises a circle.

23. The rotary valve of claim 21, wherein:
a first aperture of the two apertures of the second disc comprises a semi-circle and a second aperture of the two apertures of the second disc comprises a circle.

24. The rotary valve of claim 20, wherein each of the first and second discs comprises one aperture.

25. The rotary valve of claim 24, wherein the one aperture of each of the first and second discs is approximately the same size as the fluid conduit.

26. The rotary valve of claim 25, wherein an axis of rotation of the first and second discs is configured to be offset from a longitudinal axis of the fluid conduit.

27.-28. (canceled)

29. A rotary valve comprising:
a first valve member configured to be disposed at least partially within a fluid conduit and comprising at least one first aperture;
a second valve member configured to be disposed at least partially within the fluid conduit and comprising at least one second aperture, and
at least one of the first and second valve members being rotatable to be positioned in a plurality of positions relative to one another, and a position of the at least one first aperture relative to the at least one second aperture controlling fluid flow through the rotary valve, wherein:
the first valve member comprises a stem, one end of which includes an at least partially circular first disc extending radially outward from the stem, the first disc comprising the at least one aperture;
the second valve member comprises an annular stem, one end of which includes an at least partially circular second disc extending radially outward from the annular stem, the second disc comprising at least one aperture; and
the stem of the first valve member is received within the annular stem of the second valve member such that the first disc is adjacent the second disc.

30.-33. (canceled)

34. The rotary valve of claim 1, wherein the valve is a passive valve, wherein a pressure differential between first and second sides of the rotary valve cause at least one of the first and second valve members to rotate to be positioned in a plurality of positions relative to one another.

35.-36. (canceled)
